(19) United States
(12) Patent Application Publication
(10) Pub. No.: US 2024/0254081 A1
(43) Pub. Date: Aug. 1, 2024

(54) CATALYTIC TRYPTAMINE PROCESSES AND PRECURSORS

(71) Applicant: KARE CHEMICAL TECHNOLOGIES INC., Mississauga (CA)

(72) Inventors: Kamaluddin ABDUR-RASHID, Mississauga (CA); Kareem ABDUR-RASHID, Mississauga (CA); Wenli JIA, Toronto (CA)

(73) Assignee: KARE CHEMICAL TECHNOLOGIES INC., Mississauga, ON (CA)

(21) Appl. No.: 18/558,674

(22) PCT Filed: May 4, 2022
(86) PCT No.: PCT/CA2022/050699

§ 371 Date: Nov. 2, 2023

Related U.S. Application Data
(60) Provisional application No. 63/184,538, filed on May 5, 2021.

(51) Int. Cl. C07D 209/18

(52) U.S. Cl. CPC C07D 209/18

(57) ABSTRACT

The present disclosure relates to the use of tryptamine precursor compounds and zinc amide enolate compounds for the preparation of tryptamines. The disclosure also relates to the use of catalysts and catalytic processes for the preparation of tryptamines using the zinc amide enolate compounds and the tryptamine precursor compounds.

### CROSS-REFERENCE TO RELATED APPLICATIONS

This application claims the benefit of priority to U.S. Provisional Application No. 63/184,538 filed May 5, 2021, the contents of which are incorporated herein by reference in its entirety.

### FIELD OF THE DISCLOSURE

The present disclosure relates to the use of zinc amide enolate compounds for the preparation of tryptamines. The disclosure also relates to the use of catalysts and catalytic processes for the preparation of tryptamines using the zinc amide enolate compounds and tryptamine precursor compounds.

### BACKGROUND OF THE DISCLOSURE

Tryptamines are serotonin analogues, which can be described as derivatives of the indolamine metabolite of the amino acid tryptophan. Tryptamine itself (2-(3-indolyl)ethylamine) activates 5-HT4 receptors and regulates gastrointestinal mobility in humans (J. A. Jenkins et al. Nutrients 2016, 8, 56).

The molecular structure of substituted tryptamines contains an indole ring connected to an amino group by an ethyl linker. The indole core, ethyl linker and amino group can be further modified with other substituents.

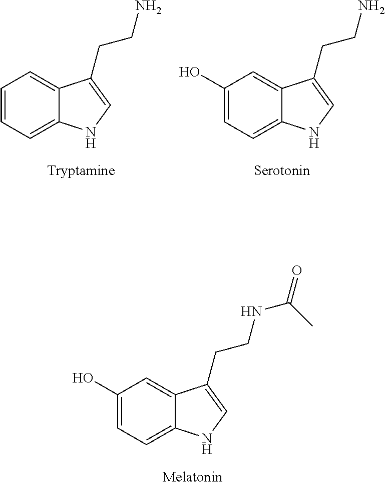 [SMILES: NCCc1c[nH]c2ccccc12 ; NCCc1c[nH]c2ccc(O)cc12 ; CC(=O)NCCc1c[nH]c2ccc(O)cc12]

The neurotransmitter serotonin (5-hydrotryptamine or 5-HT) and the sleep regulating hormone melatonin are well-known examples of substituted tryptamines (S. Young, J. Psychiatry Neurosci. 2007, 32, 394-399; R. Jockers et al. Br. J. Pharmacol. 2016, 173, 2702-2725).

The tryptamine core is present in more complex compounds such as LSD, ibogaine, mitragynine, yohimbine, cipargamin, methysergide and flovatriptan.

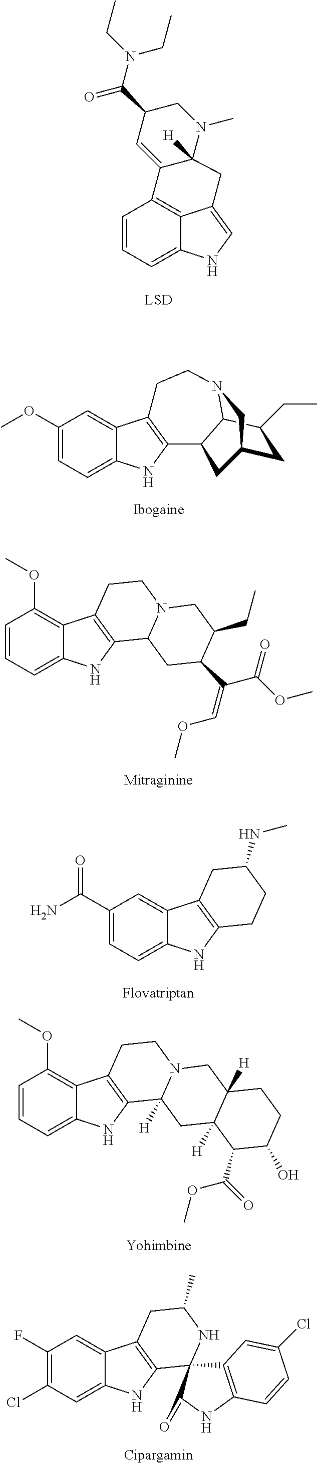

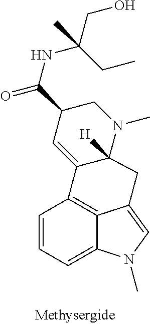

Tryptamine alkaloids are found in fungi, plants and animals. Some of these constitute traditional sources of medicines in various cultures or for neurological and psychotropic uses. These include N,N-dimethyltryptamine (DMT), 5-methoxy-N,N-dimethyltryptamine (5-MeO-DMT), bufotenin, psilocin and psilocybin (D. J. McKenna et al. J. Ethnopharmacol. 1984, 10, 195-223; J. J. H. Rucker et al. Neuropharmacology, 2018, 142, 200). Psilocybin is structurally related to other phosphorylated tryptamine natural products including norbaeocystin, baeocystin, and aeruginascin (J. Fricke et al. Angew. Chem., Int. Ed. 2017, 56, 12352-12355). On ingestion, psilocybin rapidly hydrolyses to psilocin, which is the pharmaceutically active ingredient (R. J. Dinis-Olivera Drug Metab. Rev. 2017, 49, 84)

Natural and synthetic sources of these compounds and their analogues are used as psychedelic drugs. However, they also have medicinal therapeutic uses, and several are being investigated for treating psychiatric illnesses and disorders, opioid use disorders, alcohol use disorders, sleep deprivation, anxiety disorders, major depressive disorders, and cancer-related psychiatric distress (A. C. Krugel and J. Sporn, WO 2021168082; J. P. Roiser and G. Rees Curr. Biol. 2012, 22, 231; D. E. Nichols et al. Clin. Pharmacol. Ther. 2017, 101, 209).

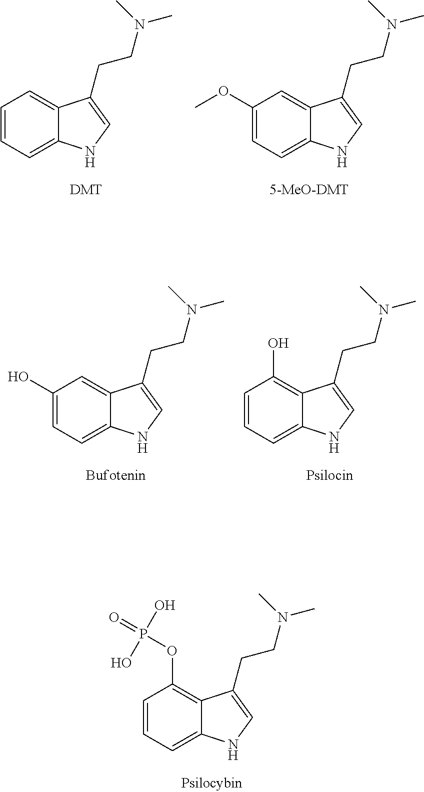

In addition to their therapeutic properties, there are increasing worldwide uses of tryptamines as recreational drugs (R. Haroz and M. I. Greenberg, Med. Clin. N. America 2005, 89, 1259-1276). The therapeutic uses and potential for abuse warrant the need for more rigorous research. Currently there is a demand for high purity compounds for investigational and therapeutic studies.

Tryptamines can be obtained from biological sources, biocatalytic processes and synthetic methods. Psilocin, psilocybin, DMT, 5-MeO-DMT and bufotenin can all be extracted from psychedelic mushrooms and plant sources. However, such supplies rely on agricultural practices, which can be difficult and inconsistent. The yields are typically low (1-2% of biomass) and some compounds, such as psilocybin decomposes easily (D. Hoffmeister et al. Chem. Eur. J. 2019, 25, 897-903).

Biosynthetic production methods are currently being developed. These typically use enzymes extracted from mushroom, plant, or animal sources. There are several reports of advances using genetically modified yeasts and microbes, along with efforts to optimize and improve production yields using generational genetic optimization techniques (A. M. Adams et al. Metabolic Engineering 2019, 56, 111-119).

Synthetic methods have been developed for several substituted tryptamines. DMT, psilocin and 5-MeO-DMT can be prepared from the reaction of the respective indole with oxalyl chloride, followed by reaction with dimethylamine and reduction of the carbonyl functionalities with lithium aluminum hydride (M. E. Speeter and W. C. Anthony J. Am. Chem. Soc. 1954, 76, 6208-6210). Phosphorylation of psilocin is used to prepare psilocybin. Bufotenin can be derived from 5-O-benzyl-DMT by catalytic hydrogenolysis.

As research advances, there is a desire for simple and economical means for the preparation of substituted tryptamines, including compound libraries, stable isotope labelled compounds and radioisotope labelled compounds. Advanced clinical studies and commercial launches of successful drug candidates will require cost-effective, environmentally friendly and scalable manufacturing processes.

### SUMMARY OF THE DISCLOSURE

The present disclosure, in some aspects, describes a new approach to the synthesis of tryptamines that focuses on the use of commercially available and stable precursors that can be transformed into the desired tryptamine products and their phosphorylated derivatives.

In various aspects, the invention relates to the use of zinc amide enolates and tryptamine precursors for the preparation of tryptamines and their derivatives using catalysts and catalytic processes. The zinc amide enolates and tryptamine precursors can be prepared and purified prior to transformation to the desired products. The indole precursors are air-stable and shelf-stable compounds that can be stored, transported and converted into the desired products on demand.

Accordingly, in some embodiments, the present invention relates to precursor compounds of Formula (I):

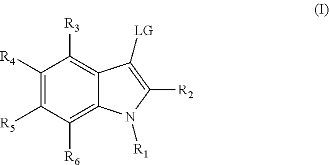

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R_{c} is a hydrogen atom or a cyclic, linear or branched alkyl, aryl or alkenyl group; LG represents any suitable leaving group, such as a halide group, sulphonate, or any other anionic leaving group;
R₂ to R₆ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₆ is optionally replaced with a heteroatom selected from the group consisting of O, S, N, P and Si, which, where possible, is optionally substituted with one or more groups.

In a general way, the compounds of Formula (I) can be prepared and isolated prior to use.

In another embodiment of the disclosure, the compound of Formula (I) is achiral.

In another embodiment of the disclosure, the compound of Formula (I) is chiral.

In another embodiment, the present disclosure relates to zinc amide enolates of Formula (II):

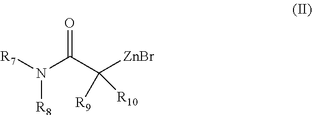

wherein,
R₇ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₇ to R₁₀ is optionally replaced with a heteroatom selected from the group consisting of O, S, N, P and Si, which, where possible, is optionally substituted with one or more groups.

In a general way, the compounds of Formula (II) can be prepared and isolated prior to use.

In another embodiment of the disclosure, the compound of Formula (II) is achiral.

In another embodiment of the disclosure, the compound of Formula (II) is chiral.

In another embodiment, the present disclosure relates to the preparation of compounds of Formula (III):

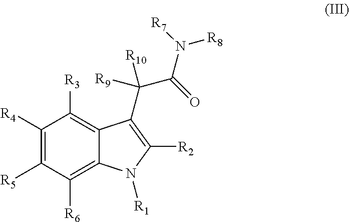

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R^{c} is a hydrogen atom or a cyclic, linear or branched alkyl, aryl or alkenyl group;
R₂ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which, where possible, is optionally substituted with one or more groups.

In a general way, the compounds of Formula (III) can be prepared and isolated prior to use.

In another embodiment of the disclosure, the compound of Formula (III) is achiral.

In another embodiment of the disclosure, the compound of Formula (III) is chiral.

In yet another embodiment, the present invention relates to the preparation of tryptamine compounds of Formula (IV):

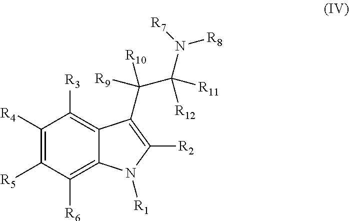

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R^{c} is a hydrogen atom or a cyclic, linear or branched alkyl, aryl or alkenyl group;
R₂ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which, where possible, is optionally substituted with one or more groups; and R₁ to R₁₂ represent hydrogen or deuterium.

In a general way, the compounds of Formula (IV) can be prepared and isolated prior to use.

In another embodiment of the disclosure, the compound of Formula (IV) is achiral.

In another embodiment of the disclosure, the compound of Formula (IV) is chiral.

In another embodiment, one or more of the carbon-12 atoms in the molecule are replaced with carbon-13.

In various embodiments of the disclosure, the transformations to which the compounds of the invention can be applied include but are not limited to catalytic and non-catalytic carbon-carbon bond forming Negishi reactions. Such carbon-carbon bond forming reactions include the use of compounds of the present disclosure to prepare tryptamine compounds.

Scheme 1 illustrates the preparation of tert-Butyl 3-(2-(dimethylamino)-2-oxoethyl)-5-methoxy-1H-indole-1-carboxylate, 2-(5-methoxy-1H-indol-3-yl)-N,N-dimethylethanamine (5-MeO-DMT) and 3-(2-(dimethylamino)ethyl)-1H-indol-5-ol (bufotenin), according to the processes of this invention. This is shown as FIG. 1.

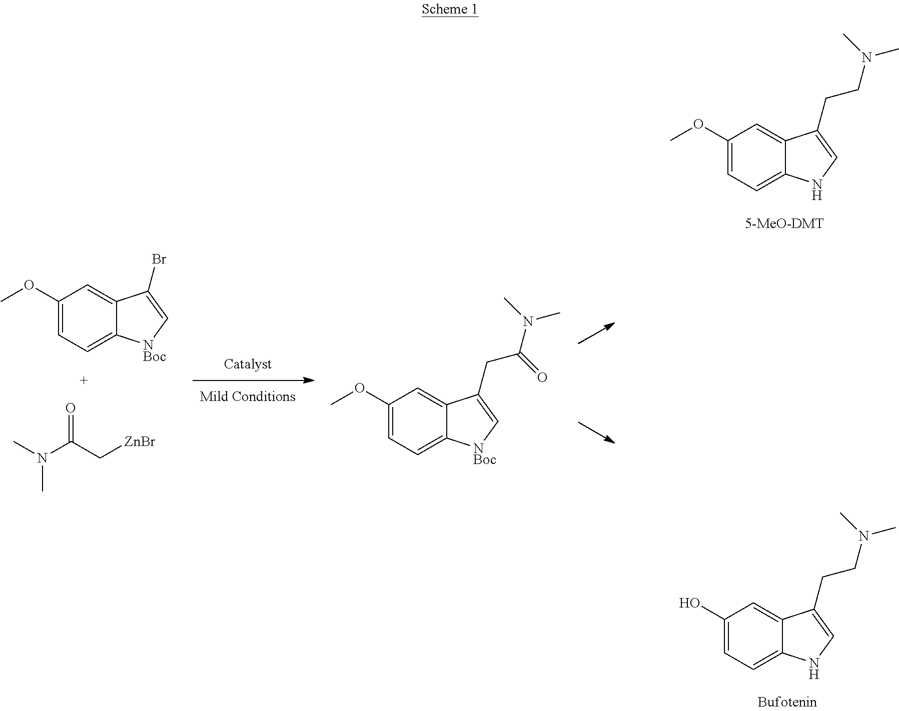

The present disclosure also includes compositions, methods of producing the compounds and compositions comprising the compounds of the invention, kits comprising any one or more of the components of the foregoing, optionally with instructions to make or use same and uses of any of the foregoing.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will be described in greater detail with reference to the following drawings, which are meant to be illustrative by certain embodiments of the invention and are not meant to limit the scope of the invention:

FIG. 1 shows the scheme for the catalytic preparation of 5-MeO-DMT and bufotenin;

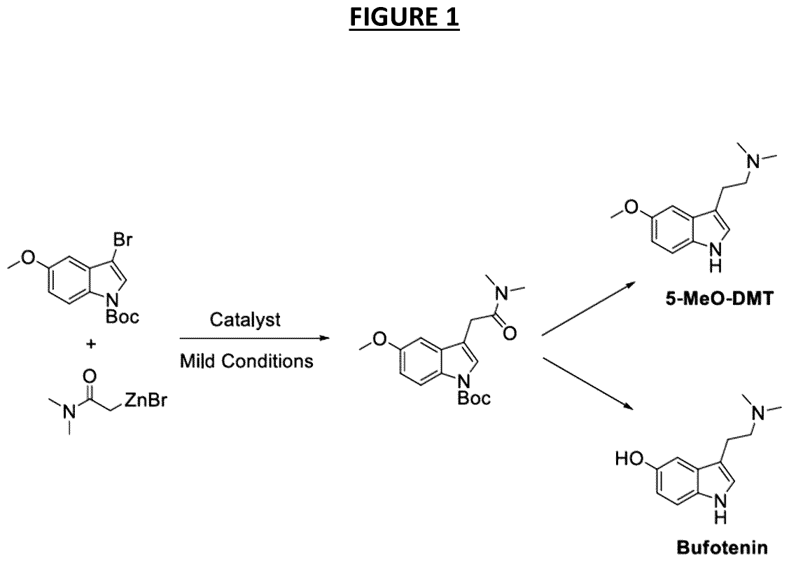

FIG. 2 shows the X-ray crystal structure of tert-butyl 5-acetoxy-3-bromo-1H-indole-1-carboxylate;

FIG. 3 shows the X-ray crystal structure of tert-butyl 3-bromo-5-methoxy-1H-indole-1-carboxylate;

FIG. 4 shows the X-ray crystal structure of tert-butyl 3-(2-(diisopropylamino)-2-oxoethyl)-5-methoxy-1H-indole-1-carboxylate;

FIG. 5 shows the X-ray crystal structure of 2-(1H-indol-3-yl)-N,N-diisopropylacetamide;

FIG. 6 shows the X-ray crystal structure of 2-(1H-indol-3-yl)-N,N-dimethylbutanamide;

FIG. 7 shows the X-ray crystal structure of 4-(2-(1H-indol-3-yl)ethyl)morpholine;

FIG. 8 shows the ¹H NMR spectrum of tert-butyl 3-(2-(dimethylamino)-2-oxoethyl)-1H-indole-1-carboxylate;

FIG. 9 shows the ¹H NMR spectrum of 2-(1H-indol-3-yl)-N,N-dimethylacetamide;

FIG. 10 shows the ¹H NMR spectrum of tert-butyl 3-(1-(dimethylamino)-1-oxopropan-2-yl)-1H-indole-1-carboxylate;

FIG. 11 shows the ¹H NMR spectrum of 2-(1H-indol-3-yl)-N,N-dimethylpropanamide;

FIG. 12 shows the ¹H NMR spectrum of 2-(1H-indol-3-yl)-N,N-dimethylbutanamide;

FIG. 13 shows the ¹H NMR spectrum of 2-(5-methoxy-1H-indol-3-yl)-N,N-dimethylacetamide;

FIG. 14 shows the ¹H NMR spectrum of 2-(4-methoxy-1H-indol-3-yl)-N,N-dimethylacetamide;

FIG. 15 shows the ¹H NMR spectrum of 2-(1H-indol-3-yl)-N,N-diisopropylacetamide;

FIG. 16 shows the ¹H NMR spectrum of N,N-diisopropyl-2-(5-methoxy-1H-indol-3-yl)acetamide;

FIG. 17 shows the ¹H NMR spectrum of tert-butyl 3-(2-morpholino-2-oxoethyl)-1H-indole-1-carboxylate;

FIG. 18 shows the ¹H NMR spectrum of 2-(1H-indol-3-yl)-1-morpholinoethanone;

FIG. 19 shows the ¹H NMR spectrum of 4-(2-(1H-indol-3-yl)ethyl)morpholine;

FIG. 20 shows the ¹H NMR spectrum of tert-butyl 5-methoxy-3-(2-morpholino-2-oxoethyl)-1H-indole-1-carboxylate;

FIG. 21 shows the ¹H NMR spectrum of 2-(5-methoxy-1H-indol-3-yl)-1-morpholinoethanone;

FIG. 22 shows the ¹H NMR spectrum of 4-(2-(5-methoxy-1H-indol-3-yl)ethyl)morpholine.

### DETAILED DESCRIPTION OF THE DISCLOSURE

### (I) Definitions

The term “(C₁-Cₚ)-alkyl” as used herein means straight and/or branched chain, saturated alkyl radicals containing one or more carbon atoms and includes (depending on the identity of “p”) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like.

The term “(C₂-Cₚ)-alkenyl” as used herein means straight and/or branched chain, unsaturated alkyl radicals containing two or more carbon atoms and one to three double bonds, and includes (depending on the identity of “p”) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like.

The term “(C₂-Cₚ)-alkynyl” as used herein means straight and/or branched chain, unsaturated alkyl radicals containing two or more carbon atoms and one to three triple bonds, and includes (depending on the identity of “p”) ethynyl, propynyl, but-1-ynyl, but-2-ynyl, but-3-ynyl, 3-methylbut-1-enyl, 3-methylpent-1-ynyl, 4-methylpent-1-ynyl, 4-methylpent-2-ynyl, penta-1,3-di-ynyl, hexyn-1-yl and the like.

The term “(C₁-Cₚ)-alkoxy” as used herein means straight and/or branched chain alkoxy group containing one or more carbon atoms and includes (depending on the identity of “p”) methoxy, ethoxy, propyloxy, isopropyloxy, t-butoxy, heptoxy, and the like.

The term “(C₃-Cₚ)-cycloalkyl” as used herein means a monocyclic, bicyclic or tricyclic saturated carbocylic group containing three or more carbon atoms and includes (depending on the identity of “p”) cyclopropyl, cyclobutyl, cyclopentyl, cyclodecyl and the like.

The term “(C₆-Cₚ)-aryl” as used herein means a monocyclic, bicyclic or tricyclic aromatic ring system containing at least one aromatic ring and 6 or more carbon atoms (and depending on the identity of “p”) and includes phenyl, naphthyl, anthracenyl, 1,2-dihydronaphthyl, 1,2,3,4-tetrahydronaphthyl, fluorenyl, indanyl, indenyl and the like.

The term “(C₅-Cₚ)-heteroaryl” as used herein means a monocyclic, bicyclic or tricyclic ring system containing one or two aromatic rings and 5 or more atoms of which, unless otherwise specified, one, two, three, four or five are heteromoieties independently selected from N, NH, N(alkyl), O and S and depending on the value of “p” includes thienyl, furyl, pyrrolyl, pyrididyl, indolyl, quinolyl, isoquinolyl, tetrahydroquinolyl, benzofuryl, benzothienyl and the like.

The term “halo” or “halogen” as used herein means chloro, fluoro, bromo or iodo.

The term “fluoro-substituted” as used herein means that at least one, including all, of the hydrogens on the referenced group is replaced with fluorine.

The suffix “ene” added on to any of the above groups means that the group is divalent, i.e. inserted between two other groups.

The term “ring system” as used herein refers to a carbon-containing ring system, that includes monocycles, fused bicyclic and polycyclic rings, bridged rings and metalocenes. Where specified, the carbons in the rings may be substituted or replaced with heteroatoms.

The term “leaving group” as used herein refers to a group capable of being displaced from a compound when the compound undergoes reaction with a nucleophile.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. For instance, “including” also encompasses “including but not limited to”. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

The present disclosure relates to precursors compounds of Formula (I):

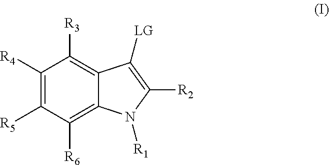

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R^{c} is a hydrogen atom or a cyclic, linear or branched alkyl, aryl or alkenyl group;
LG represents any suitable leaving group, such as a halide group, sulphonate, or any other anionic leaving group; and
R₂ to R₆ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₆ is optionally replaced with a heteroatom selected from the group consisting of O, S, N, P and Si, which, where possible, is optionally substituted with one or more groups.

In a general way, the compounds of Formula (I) can be prepared and isolated prior to use.

In another embodiment of the disclosure, the compound of Formula (I) is achiral.

In another embodiment of the disclosure, the compound of Formula (I) is chiral.

In one embodiment, R₁ represents hydrogen, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, —(C═O)—O—(C₁-C₂₀)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₂₀)-alkyl, (C₂-C₂₀)-alkenyl, (C₂-C₂₀)-alkynyl, (C₃-C₂₀)-cycloalkyl, or (C₆-C₂₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, —(C═O)—O—(C₁-C₁₀)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₁₀)-alkyl, (C₂-C₁₀)-alkenyl, (C₂-C₁₀)-alkynyl, (C₃-C₁₀)-cycloalkyl, or (C₆-C₁₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, —(C═O)—O—(C₁-C₆)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₆)-alkyl, (C₂-C₆)-alkenyl, (C₂-C₆)-alkynyl, (C₃-C₇)-cycloalkyl, or (C₆)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₆)-alkyl, (C₂-C₆)-alkenyl, (C₂-C₆)-alkynyl, (C₃-C₇)-cycloalkyl, phenyl, or —C(═O)—(C₁-C₆)-alkyl, each of which are optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ is a nitrogen protecting group such as a phosphinyl group, a phosphoryl group, a sulfenyl group, a sulfonyl group, or a silyl group (such as TMS, TIPS, TBDMS).

In one embodiment, LG represents any suitable leaving group, such as a halide group, sulphonate, or any other anionic leaving group. In one embodiment, LG is chloro, bromo or iodo. In another embodiment, LG is mesylate, triflate or tosylate.

In one embodiment, R₂ to R₆ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₂ to R₆ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₆ represent hydrogen, deuterium, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, or two adjacent or geminal groups are bonded together to form an optionally substituted ring, each of which is optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₂ to R₆ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₆ represent hydrogen, deuterium, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, or two adjacent or geminal groups are bonded together to form an optionally substituted ring, each of which is optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₂ to R₆ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₆ represent hydrogen, deuterium, (C₁-C₆)-alkyl, (C₂-C₆)-alkenyl, (C₂-C₆)-alkynyl, (C₃-C₇)-cycloalkyl, (C₆)-aryl, or (C₅-C₆)-heteroaryl.

The present disclosure also relates to a tryptamine precursors of Formula (I), wherein one or more of the carbon-12 atoms are replaced with carbon-13.

The present disclosure also relates to zinc amide enolates of Formula (II):

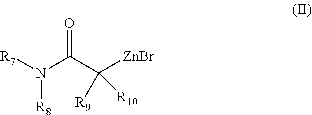

wherein,
R₇ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted 5-10-membered carboyclic or heterocyclic ring, or an acyl group, possibly substituted, and one or more of the carbon atoms in the alkyl, alkenyl, alkynyl, cycloalkyl, aryl, heteroaryl or acyl groups of R₇ to R₁₀ is optionally replaced with a heteroatom selected from the group consisting of O, S, N, P and Si, which, where possible, is optionally substituted with one or more groups.

In one embodiment, R₇ to R₁₀ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₇ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₇ to R₁₀ represent hydrogen, deuterium, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, or two adjacent or geminal groups are bonded together to form an optionally substituted ring, each of which is optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₇ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₇ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, or two adjacent or geminal groups are bonded together to form an optionally substituted ring, each of which is optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₇ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₇ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, or (C₅-C₆)-heteroaryl.

In one embodiment, R₇ and R₈ are joined together, along with the nitrogen atom to which they are attached, to form a 5-8-membered carbocyclic or heterocyclic ring. In one embodiment, the 5-8-membered ring is optionally substituted with halogen, oxo (C═O), OH, optionally substituted phenyl or (C₁-C₆)-alkyl

In a general way, the compounds of Formula (II) can be prepared and isolated prior to use.

In another embodiment of the disclosure, the compound of Formula (II) is achiral.

In another embodiment of the disclosure, the compound of Formula (II) is chiral.

The present disclosure also relates to compounds of Formula (III):

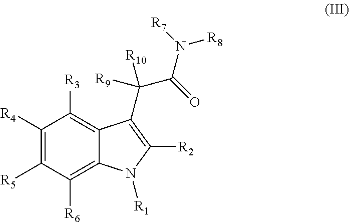

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R^{c} is a hydrogen atom or a cyclic, linear or branched alkyl, aryl or alkenyl group;
R₂ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which, where possible, is optionally substituted with one or more groups.

In one embodiment, R₁ represents hydrogen, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, —(C═O)—O—(C₁-C₂₀)-alkyl, OR^{c}, or NR^{c}₂, each of which are optionally substituted with halogen, OH, or (C₁-C₆)-alkyl, and wherein R^{c} is hydrogen, (C₁-C₂₀)-alkyl, (C₂-C₂₀)-alkenyl, (C₂-C₂₀)-alkynyl, (C₃-C₂₀)-cycloalkyl, or (C₆-C₂₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, —(C═O)—O—(C₁-C₁₀)-alkyl, OR^{c}, or NR^{c}₂, each of which are optionally substituted with halogen, OH, or (C₁-C₆)-alkyl, and wherein R^{c} is hydrogen, (C₁-C₁₀)-alkyl, (C₂-C₁₀)-alkenyl, (C₂-C₁₀)-alkynyl, (C₃-C₁₀)-cycloalkyl, or (C₆-C₁₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, —(C═O)—O—(C₁-C₆)-alkyl, OR^{c}, or NR^{c}₂, each of which are optionally substituted with halogen, OH, or (C₁-C₆)-alkyl, and wherein R^{c} is hydrogen, (C₁-C₆)-alkyl, (C₂-C₆)-alkenyl, (C₂-C₆)-alkynyl, (C₃-C₇)-cycloalkyl, or (C₆)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₆)-alkyl, (C₂-C₆)-alkenyl, (C₂-C₆)-alkynyl, (C₃-C₇)-cycloalkyl, phenyl, or —C(═O)—(C₁-C₆)-alkyl, each of which are optionally substituted with halogen, OH, or (C₁-C₆)-alkyl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₂ to R₁₀ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R_{\10} is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, or (C₅-C₆)-heteroaryl.

In a general way, the compounds of Formula (III) can be prepared and isolated prior to use.

In another embodiment of the disclosure, the compound of Formula (III) is achiral.

In another embodiment of the disclosure, the compound of Formula (III) is chiral.

In yet another embodiment, the present disclosure relates to compounds of Formula (IV):

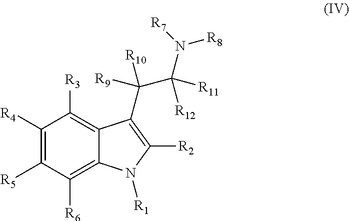

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R^{c} is a hydrogen atom or a cyclic, linear or branched alkyl, aryl or alkenyl group;
R₂ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which, where possible, is optionally substituted with one or more groups; and
R₁ to R₁₂ represent hydrogen or deuterium.

In one embodiment, R₁ represents hydrogen, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, —(C═O)—O—(C₁-C₂₀)-alkyl, OR^{c}, or NR^{c}₂, each of which are optionally substituted with halogen, OH, or (C₁-C₆)-alkyl, and wherein R^{c} is hydrogen, (C₁-C₂₀)-alkyl, (C₂-C₂₀)-alkenyl, (C₂-C₂₀)-alkynyl, (C₃-C₂₀)-cycloalkyl, or (C₆-C₂₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, —(C═O)—O—(C₁-C₁₀)-alkyl, OR^{c}, or NR^{c}₂, each of which are optionally substituted with halogen, OH, or (C₁-C₆)-alkyl, and wherein R^{c} is hydrogen, (C₁-C₁₀)-alkyl, (C₂-C₁₀)-alkenyl, (C₂-C₁₀)-alkynyl, (C₃-C₁₀)-cycloalkyl, or (C₆-C₁₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, —(C═O)—O—(C₁-C₆)-alkyl, OR^{c}, or NR^{c}₂, each of which are optionally substituted with halogen, OH, or (C₁-C₆)-alkyl, and wherein R^{c} is hydrogen, (C₁-C₆)-alkyl, (C₂-C₆)-alkenyl, (C₂-C₆)-alkynyl, (C₃-C₇)-cycloalkyl, or (C₆)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₁ represents hydrogen, (C₁-C₆)-alkyl, (C₂-C₆)-alkenyl, (C₂-C₆)-alkynyl, (C₃-C₇)-cycloalkyl, phenyl, or —C(═O)—(C₁-C₆)-alkyl, each of which are optionally substituted with halogen, OH, or (C₁-C₆)-alkyl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

In one embodiment, R₂ to R₁₀ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R_{\10} is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, or (C₅-C₆)-heteroaryl.

In a general way, the compounds of Formula (IV) can be prepared and isolated prior to use.

In another embodiment of the disclosure, the compound of Formula (IV) is achiral.

In another embodiment of the disclosure, the compound of Formula (IV) is chiral.

In another embodiment, one or more of the carbon-12 atoms in the molecule are replaced with carbon-13.

In another embodiment, the present disclosure relates to compounds of Formula (V), Formula (VI), Formula (VII) and Formula (VIII):

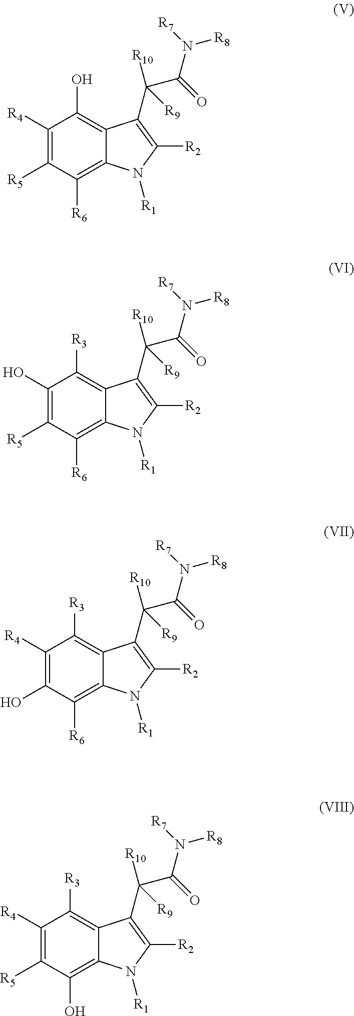

wherein,
R₁ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, or a carbamate group, possibly substituted, or an OR^{c} group or an NR^{c}₂ group, possibly substituted, in which R^{c} is a hydrogen atom or a cyclic, linear or branched alkyl, aryl or alkenyl group;
R₂ to R₁₀ represent hydrogen, deuterium, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or two adjacent or geminal groups are bonded together to form an optionally substituted ring, or an acyl group, possibly substituted, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which, where possible, is optionally substituted with one or more groups.

In one embodiment, R₁ represents hydrogen, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, —(C═O)—O—(C₁-C₂₀)-alkyl, OR^{c}, or NR^{c}₂, each of which are optionally substituted with halogen, OH, or (C₁-C₆)-alkyl, and wherein R^{c} is hydrogen, (C₁-C₂₀)-alkyl, (C₂-C₂₀)-alkenyl, (C₂-C₂₀)-alkynyl, (C₃-C₂₀)-cycloalkyl, or (C₆-C₂₀)-aryl.

In one embodiment, R₁ represents hydrogen, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)-(C₁-C₂₀)-alkyl, —(C═O)—O—(C₁-C₂₀)-alkyl, OR^{c}, or NR^{c}₂, each of which are optionally substituted with halogen, OH, or (C₁-C₆)-alkyl, and wherein R^{c} is hydrogen, (C₁-C₁₀)-alkyl, (C₂-C₁₀)-alkenyl, (C₂-C₁₀)-alkynyl, (C₃-C₁₀)-cycloalkyl, or (C₆-C₁₀)-aryl.

In one embodiment, R₁ represents hydrogen, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, —(C═O)—O—(C₁-C₆)-alkyl, OR^{c}, or NR^{c}₂, each of which are optionally substituted with halogen, OH, or (C₁-C₆)-alkyl, and wherein R^{c} is hydrogen, (C₁-C₆)-alkyl, (C₂-C₆)-alkenyl, (C₂-C₆)-alkynyl, (C₃-C₇)-cycloalkyl, or (C₆)-aryl.

In one embodiment, R₁ represents hydrogen, (C₁-C₆)-alkyl, (C₂-C₆)-alkenyl, (C₂-C₆)-alkynyl, (C₃-C₇)-cycloalkyl, phenyl, or —C(═O)—(C₁-C₆)-alkyl, each of which are optionally substituted with halogen, OH, or (C₁-C₆)-alkyl.

In one embodiment, R₂ to R₁₀ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

In one embodiment, R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, or (C₅-C₆)-heteroaryl

In a general way, the compounds of Formula (V), Formula (VI), Formula (VII) and Formula (VIII) can be prepared and isolated prior to use.

In another embodiment of the disclosure, the compounds of Formula (V), Formula (VI), Formula (VII) and Formula (VIII) are achiral.

In another embodiment of the disclosure, the compounds of Formula (V), Formula (VI), Formula (VII) and Formula (VIII) are chiral.

In another embodiment, one or more of the carbon-12 atoms in the molecule are replaced with carbon-13.

### (III) Processes of the Disclosure

The present disclosure also relates to processes for the preparation of compounds of Formula (III):

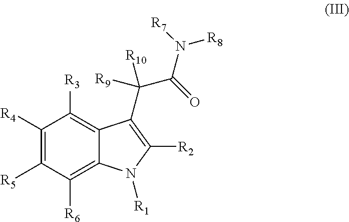

by contacting a compound of Formula (I):

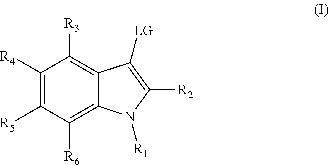

with a compound of Formula (II):

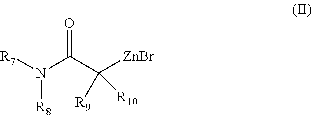

in the presence of a suitable catalyst,
wherein the variables R₁-R₁₀ and LG are as defined above.

In some aspects, the transformation of a compound of Formula (I) and Formula (II) to a compound of Formula (III) requires a suitable catalyst. Suitable catalysts include but are not limited to transition metal salts and complexes, such as compounds of palladium, nickel, iron, ruthenium, cobalt, rhodium, iridium and copper.

In some aspects, the catalysts are chiral and can facilitate asymmetric carbon-carbon bond forming reactions.

The disclosure also relates to processes for the catalytic and non-catalytic conversion of compounds of Formula (III):

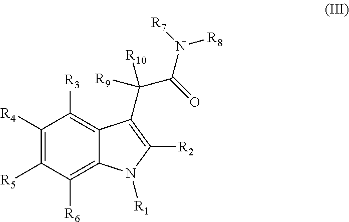

to compounds of Formula (IV):

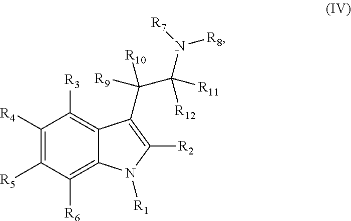

wherein the variables R₁-R₁₂ and LG are as defined above.

Carbon-carbon bond forming reactions for the preparation of compounds of Formula (III) include but are not limited to catalytic and non-catalytic Negishi reactions.

Reactions for the preparation of compounds of Formula (IV) include but are not limited to catalytic and non-catalytic reduction and hydrogenation reactions. Suitable reducing agents include borohydrides, borodeuterides, aluminohydrides, aluminodeuterides, silanes, boranes, hydrogen gas and deuterium gas.

In some embodiments of the disclosure, the catalytic system characterizing the process of the instant invention may comprise a base. In some embodiments, said base can be any conventional base. In some embodiments, non-limiting examples include: organic non-coordinating bases such as DBU, an alkaline or alkaline-earth metal carbonate, a carboxylate salt such as sodium or potassium acetate, or an alcoholate or hydroxide salt.

Preferred bases are the alcoholate or hydroxide salts selected from the group consisting of the compounds of formula (RO)₂M′ and ROM″, wherein M′ is an alkaline-earth metal, M″ is an alkaline metal and R stands for hydrogen or a linear or branched alkyl group.

The catalyst can be added to the reaction medium in a large range of concentrations. As non-limiting examples, one can cite as catalyst concentration values ranging from 0.001% to 50%, relative to the amount of substrate, thus representing respectively a substrate/catalyst (S/cat) ratio of 100,000 to 2. Preferably, the complex concentration will be comprised between 0.01% and 10%, i.e. a S/cat ratio of 10,000 to 10 respectively. In some preferred embodiments, there will be used concentrations in the range of 0.1 to 5%, corresponding to a S/cat ratio of 1000 to 20 respectively.

If required, useful quantities of base, added to the reaction mixture, may be comprised in a relatively large range. In some embodiments, non-limiting examples include: ranges between 1 to 100 molar equivalents relative to the substrate. However, it should be noted that it is also possible to add a small amount of base (e.g. base/substrate=1 to 3) to achieve high yields.

In the processes of this disclosure, the catalytic reaction can be carried out in the presence or absence of a solvent. When a solvent is required or used for practical reasons, then any solvent currently used in catalytic reactions can be used for the purposes of the invention. Non-limiting examples include aromatic solvents such as benzene, toluene or xylene, hydrocarbon solvents such as hexane or cyclohexane, ethers such as tetrahydrofuran, or yet primary or secondary alcohols, or water, or mixtures thereof. A person skilled in the art is well able to select the solvent most convenient in each case to optimize the catalytic reaction.

The temperature at which the catalytic reaction can be carried out is comprised between −30° C. and 200° C., more preferably in the range of between 0° C. and 100° C. Of course, a person skilled in the art is also able to select the preferred temperature.

Standard catalytic conditions, as used herein, typically implies the mixture of the substrate with the catalyst with or without a base, possibly in the presence of a solvent, and then treating such a mixture with the desired reactant at a chosen temperature in air or under an inert atmosphere of nitrogen or argon gas. Varying the reaction conditions, including for example, catalyst, temperature, solvent and reagent, to optimize the yield of the desired product would be well within the abilities of a person skilled in the art.

The present disclosure is described in the following Examples, which are set forth to aid in the understanding of the invention, and should not be construed to limit in any way the scope of the invention as defined in the claims which follow thereafter.

### EXAMPLES

The disclosure will now be described in further details by way of the following examples, wherein the temperatures are indicated in degrees centigrade and the abbreviations have the usual meaning in the art.

All the procedures described hereafter have been carried out under an inert atmosphere unless stated otherwise. All preparations and manipulations under air-free conditions were carried out under N₂ or Ar atmospheres with the use of standard Schlenk, vacuum line and glove box techniques in dry, oxygen-free solvents. Deuterated solvents were degassed and dried over activated molecular sieves. NMR spectra were recorded on a 400 MHz spectrometer (400 MHz for ¹H, 100 MHz for ¹³C, 376 MHz for ¹⁹F and 162 MHz for ³¹P). All ³¹P chemical shifts were measured relative to 85% H3PO4 as an external reference. ¹H and ¹³C chemical shifts were measured relative to partially deuterated solvent peaks but are reported relative to tetramethylsilane.

### Example 1. Preparation of 1H-indol-4-yl acetate

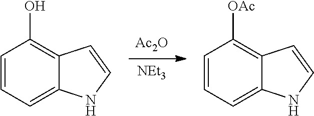 [SMILES: "Ac2O" ; Oc1cccc2[nH]ccc12 ; CC(=O)Oc1cccc2[nH]ccc12 ; "NEt3"]

Acetic anhydride (3.27 g, 32 mmol) was added slowly to a mixture of 4-hydroxyindole (3.88 g, 29 mmol) and triethylamine (4.4 g, 44 mmol) in dichloromethane (50 ml) at room temperature. The reaction was stirred for 3 hours, then water (30 ml) added. The phases were separated, and the aqueous layer was extracted with dichloromethane (2×15 ml). The combined organic layer was washed with water (50 ml), then brine (20 ml) and dried (MgSO₄). The solvent was evaporated under reduced pressure and the residue eluted through a silica gel pad using hexanes/ethyl acetate (2:1) as eluent. The solvent was removed, and the residue was dried under vacuum to give the product as a white crystalline solid. Yield=4.75 g.

### Example 2. Preparation of tert-Butyl 4-acetoxy-1H-indole-1-carboxylate

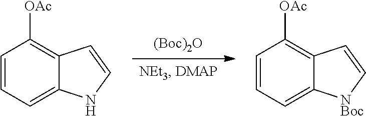

A solution of (Boc)₂O(1.3 g, 6.0 mmol) in dichloromethane (5 ml) was added to a mixture of 1H-indol-4-yl acetate (1.33 g, 5.7 mmol), triethylamine (1.15 g, 11.4 mmol), DMAP (2 mg) in dichloromethane (10 ml) and the reaction stirred overnight at room temperature. It was quenched with saturated NaHCO₃ solution (20 ml) and the phases separated. The aqueous layer was extracted with dichloromethane (2×10 ml). The combined organic portion was washed with brine and dried over MgSO₄. The solvent was removed, and the residue eluted through a silica gel pad using ethyl acetate/hexanes (1:3) as eluent. The solvent was removed, and the residue was dried under vacuum to give the product as a colourless oil. Yield=1.48 g.

### Example 3. Preparation of tert-Butyl 4-acetoxy-3-bromo-1H-indole-1-carboxylate

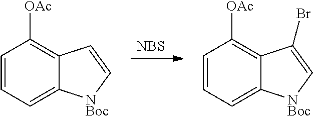

NBS (3.06 g, 17.2 mmol) was added to a mixture of tert-butyl 4-acetoxy-1H-indole-1-carboxylate (4.5 g, 16.4 mmol) and NH₄Cl (5 mg) in dichloromethane (100 ml) and the reaction stirred overnight at room temperature. Water (50 ml) was added, and the phases separated. The combined organic portion was washed with brine (20 ml), then water (20 ml), then dried over MgSO₄ and filtered. The solvent was removed, and the residue was eluted through a silica gel pad using EA/CH₂Cl₂/hexanes (1:2:10) as eluent. The solvent was removed, and the residue dried under vacuum to give the product as a white crystalline solid. Yield=6.0 g.

### Example 4. Preparation of 4-(tert-butyldimethylsilyloxy)-1H-indole

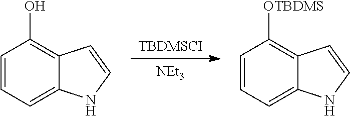 [SMILES: "TBDMSCI" ; Oc1cccc2[nH]ccc12 ; CC(C)(C)[Si](C)(C)Oc1cccc2[nH]ccc12 ; "NEt3"]

Triethylamine (2.28 g, 22.5 mmol) was added to a solution of 4-hydroxyindole (2.0 g, 15.0 mmol) in dichloromethane (10 ml), followed by TBDMSCl (2.26 g, 15.0 mmol) and the mixture was stirred at room temperature for 20 hours. The solvent was removed under reduced pressure and hexanes/ether (5:2, 20 ml) was added. The mixture was stirred for 30 minutes, then filtered through a pad of silica gel. The filtrate was evaporated to dryness to give the product as an off-white solid. Yield=3.68 g.

### Example 5. Preparation of tert-butyl 4-(tert-butyldimethylsilyloxy)-1H-indole-1-carboxylate

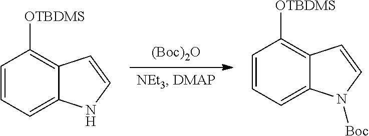

Triethylamine (3.04 g, 30 mmol) was added to a solution of 4-(tert-butyldimethylsilyloxy)-1H-indole (3.1 g, 12.5 mmol) in dichloromethane (20 ml) and di-tert-butyl decarbonate (3.27 g, 15 mmol) added, followed by DMAP (0.08 g, 0.65 mmol). The mixture was stirred for 24 hours with venting of the evolved gas through a bubbler. The reaction was evaporated to dryness and the residue was eluted through a pad of silica gel using hexanes/ether (7:1). The filtrate was evaporated to dryness to give the product as a colourless oil. Yield=4.29 g.

### Example 6. Preparation of tert-butyl 3-bromo-4-(tert-butyldimethylsilyloxy)-1H-indole-1-carboxylate

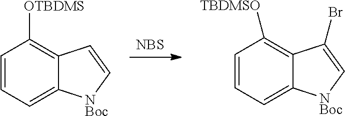

NBS (0.282 g, 1.58 mmol) was added to a mixture of tert-butyl 4-(tert-butyldimethylsilyloxy)-1H-indole-1-carboxylate (0.5 g, 1.44 mmol) in dichloromethane (10 ml) and the reaction stirred overnight at room temperature. It was quenched with saturated NaHCO₃ solution (10 ml) and the phases separated. The aqueous layer was extracted with dichloromethane (2×10 ml) and the combined organic portion was washed water (10 ml), then dried over MgSO₄ and filtered. The solvent was removed, and the residue was eluted through a silica gel pad using CH₂Cl₂/hexanes (1:3) as eluent. The solvent was removed, and the residue dried under vacuum to give the product as a colourless oil. Yield=0.42 g.

### Example 7. Preparation of tert-butyl 4-methoxy-1H-indole-1-carboxylate

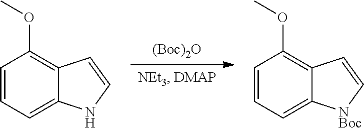

Triethylamine (2.75 g, 27.2 mmol) was added to a solution of 4-methoxyindole (2.0 g, 13.6 mmol) in dichloromethane (20 ml) and di-tert-butyl decarbonate (3.0 g, 13.7 mmol) added, followed by DMAP (0.07 g, 0.54 mmol). The mixture was stirred for 16 hours with venting of the evolved gas through a bubbler. The reaction was evaporated to dryness and the residue was eluted through a pad of silica gel using hexanes/ethylacetate. The filtrate was evaporated to dryness to give the product as a colourless oil. Yield=3.17 g.

### Example 8. Preparation of tert-butyl 3-bromo-4-methoxy-1H-indole-1-carboxylate

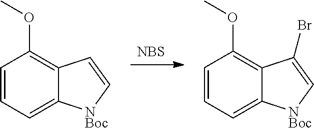

NBS (9.0 g, 50.6 mmol) was added to a mixture of tert-butyl 4-methoxy-1H-indole-1-carboxylate (11.5 g, 46.5 mmol) and NH₄Cl (20 mg) in dichloromethane (200 ml), THE (10 ml) and DMF (4 drops) and the reaction stirred overnight at room temperature. It was quenched with saturated NaHCO₃ solution (100 ml) and the phases separated. The aqueous layer was extracted with dichloromethane (2×30 ml) and the combined organic portion was washed with brine (50 ml), then water (50 ml), then dried over MgSO₄ and filtered. The solvent was removed, and the residue was eluted through a silica gel pad using CH₂Cl₂/hexanes (31) as eluent. The solvent was removed, and the residue dried under vacuum to give the product as a white crystalline solid. Yield=11.2 g.

### Example 9. Preparation of 1H-indol-5-yl acetate

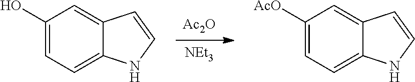 [SMILES: "Ac2O" ; Oc1ccc2[nH]ccc2c1 ; CC(=O)Oc1ccc2[nH]ccc2c1 ; "NEt3"]

Acetic anhydride (4.4 g, 43 mmol) was added slowly to a mixture of 5-hydroxyindole (5.2 g, 39 mmol) and triethylamine (5.9 g, 58 mmol) in dichloromethane (50 ml) at room temperature. The reaction was stirred for 3 hours, then water (30 ml) added. The phases were separated, and the aqueous layer was extracted with dichloromethane (2×15 ml). The combined organic layer was washed with water (100 ml), then brine (20 ml) and dried (MgSO₄). The solvent was evaporated under reduced pressure and the residue eluted through a silica gel pad using hexanes/ethyl acetate (2:1) as eluent. The solvent was removed, and the residue was dried under vacuum to give the product as a white crystalline solid. Yield=6.75 g.

### Example 10. Preparation of tert-Butyl 5-acetoxy-1H-indole-1-carboxylate

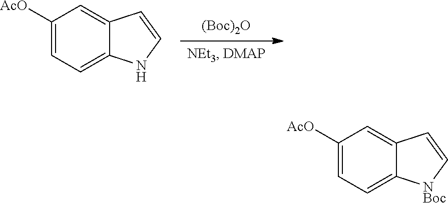

A solution of (Boc)₂O(9.25 g, 42 mmol) in dichloromethane (20 ml) was added to a mixture of 1H-indol-5-yl acetate (6.75 g, 38 mmol), triethylamine (7.78 g, 77 mmol), DMAP (5 mg) in dichloromethane (70 ml) and the reaction stirred overnight at room temperature. It was quenched with saturated NaHCO₃ solution (50 ml) and the phases separated. The aqueous layer was extracted with dichloromethane (2×20 ml). The combined organic portion was washed with brine and dried over MgSO₄. The solvent was removed, and the residue eluted through a silica gel pad using ethyl acetate/hexanes (1:3) as eluent. The solvent was removed, and the residue was dried under vacuum to give the product as a pale-yellow oil. Yield=10.63 g.

### Example 11. Preparation of tert-Butyl 5-acetoxy-3-bromo-1H-indole-1-carboxylate

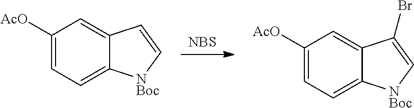

NBS (6.83 g, 38.4 mmol) was added to a mixture of tert-butyl 5-acetoxy-1H-indole-1-carboxylate (10.0 g, 36.3 mmol) and NH₄Cl (0.2 g) in dichloromethane (100 ml) and the reaction stirred overnight at room temperature. Water (40 ml) was added, and the phases separated. The combined organic portion was washed with brine (30 ml), then water (20 ml), then dried over MgSO₄ and filtered. The solvent was removed, and the residue was eluted through a silica gel pad using EA/CH₂Cl₂/hexanes (1:2:10) as eluent. The solvent was removed, and the residue dried under vacuum to give the product as a white crystalline solid. Yield=10.29 g.

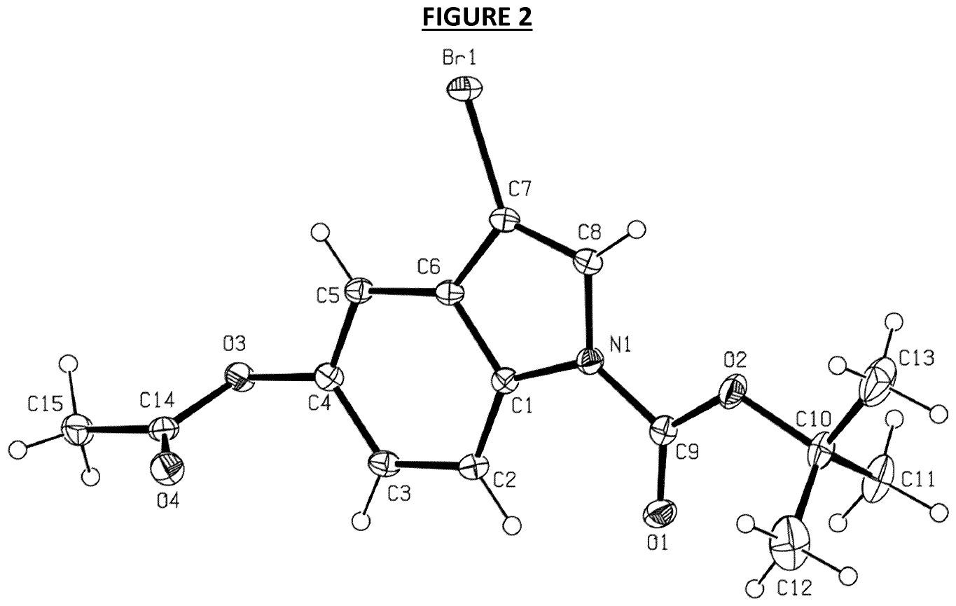

FIG. 2 shows the X-ray crystal structure of tert-Butyl 5-acetoxy-3-bromo-1H-indole-1-carboxylate.

### Example 12. Preparation of 5-(tert-butyldimethylsilyloxy)-1H-indole

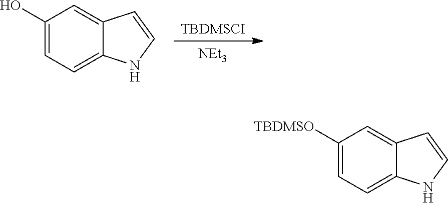 [SMILES: "TBDMSCI" ; Oc1ccc2[nH]ccc2c1 ; CC(C)(C)[Si](C)(C)Oc1ccc2[nH]ccc2c1 ; "NEt3"]

Triethylamine (2.28 g, 22.5 mmol) was added to a solution of 5-hydroxyindole (2.0 g, 15.0 mmol) in dichloromethane (10 ml), followed by TBDMSCl (2.26 g, 15.0 mmol) and the mixture was stirred at room temperature for 20 hours. The solvent was removed under reduced pressure and hexanes/ether (5:2, 20 ml) was added. The mixture was stirred for 30 minutes, then filtered through a pad of silica gel. The filtrate was evaporated to dryness to give the product as an off-white solid. Yield=4.2 g.

### Example 13. Preparation of tert-butyl 5-(tert-butyldimethylsilyloxy)-1H-indole-1-carboxylate

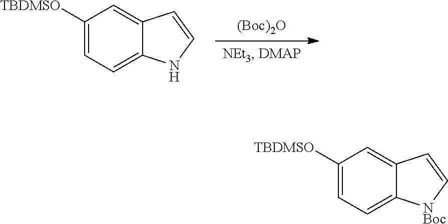

Triethylamine (3.04 g, 30 mmol) was added to a solution of 5-(tert-butyldimethylsilyloxy)-1H-indole (3.1 g, 12.5 mmol) in dichloromethane (20 ml) and di-tert-butyl decarbonate (3.27 g, 15 mmol) added, followed by DMAP (0.08 g, 0.65 mmol). The mixture was stirred for 24 hours with venting of the evolved gas through a bubbler. The reaction was evaporated to dryness and the residue was eluted through a pad of silica gel using hexanes/ether (7:1). The filtrate was evaporated to dryness to give the product as a colourless oil. Yield=4.1 g.

### Example 14. Preparation of tert-butyl 3-bromo-5-(tert-butyldimethylsilyloxy)-1H-indole-1-carboxylate

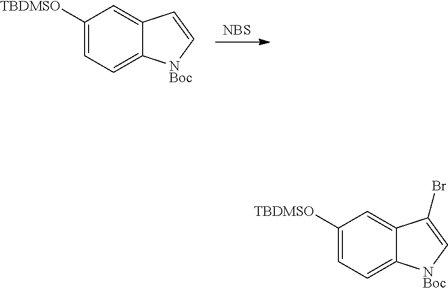

NBS (0.282 g, 1.58 mmol) was added to a mixture of tert-butyl 5-(tert-butyldimethylsilyloxy)-1H-indole-1-carboxylate (0.5 g, 1.44 mmol) in dichloromethane (10 ml) and the reaction stirred overnight at room temperature. It was quenched with saturated NaHCO₃ solution (10 ml) and the phases separated. The aqueous layer was extracted with dichloromethane (2×10 ml) and the combined organic portion was washed water (10 ml), then dried over MgSO₄ and filtered. The solvent was removed, and the residue was eluted through a silica gel pad using CH₂Cl₂/hexanes (1:3) as eluent. The solvent was removed, and the residue dried under vacuum to give the product as a colourless oil. Yield=0.38 g.

### Example 15. Preparation of tert-butyl 5-methoxy-1H-indole-1-carboxylate

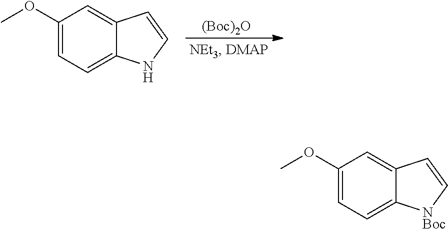 [SMILES: "(Boc)2O" ; COc1ccc2[nH]ccc2c1 ; COc1ccc2c(ccn2C(=O)OC(C)(C)C)c1]

A solution of (Boc)₂O(15.0 g, 68.6 mmol) in dichloromethane (20 ml) was added to a mixture of 5-methoxy-1H-indole 10.0 g, 68 mmol), triethylamine (13.7 g, 136 mmol), DMAP (80 mg) in dichloromethane (100 ml) and the reaction stirred overnight at room temperature. It was quenched with saturated NaHCO₃ solution (60 ml) and the phases separated. The aqueous layer was extracted with dichloromethane (2×30 ml). The combined organic portion was washed with brine and dried over MgSO₄. The solvent was removed, and the residue eluted through a silica gel pad using CH₂Cl₂/hexanes (1:1) as eluent. The solvent was removed, and the residue was dried under vacuum to give the product as a white crystalline solid. Yield=16.7 g.

### Example 16. Preparation of tert-butyl 3-bromo-5-methoxy-1H-indole-1-carboxylate

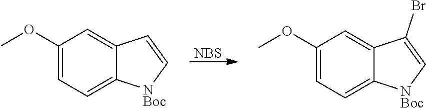

NBS (9.0 g, 50.6 mmol) was added to a mixture of tert-butyl 5-methoxy-1H-indole-1-carboxylate (11.5 g, 46.5 mmol) and NH₄Cl (20 mg) in dichloromethane (200 ml), THE (10 ml) and DMF (4 drops) and the reaction stirred for one hour at room temperature. It was quenched with saturated NaHCO₃ solution (100 ml) and the phases separated. The aqueous layer was extracted with dichloromethane (2×30 ml) and the combined organic portion was washed with brine (50 ml), then water (50 ml), then dried over MgSO₄ and filtered. The solvent was removed, and the residue was eluted through a silica gel pad using CH₂Cl₂/hexanes (31) as eluent. The solvent was removed, and the residue dried under vacuum to give the product as a white crystalline solid. Yield=13.7 g.

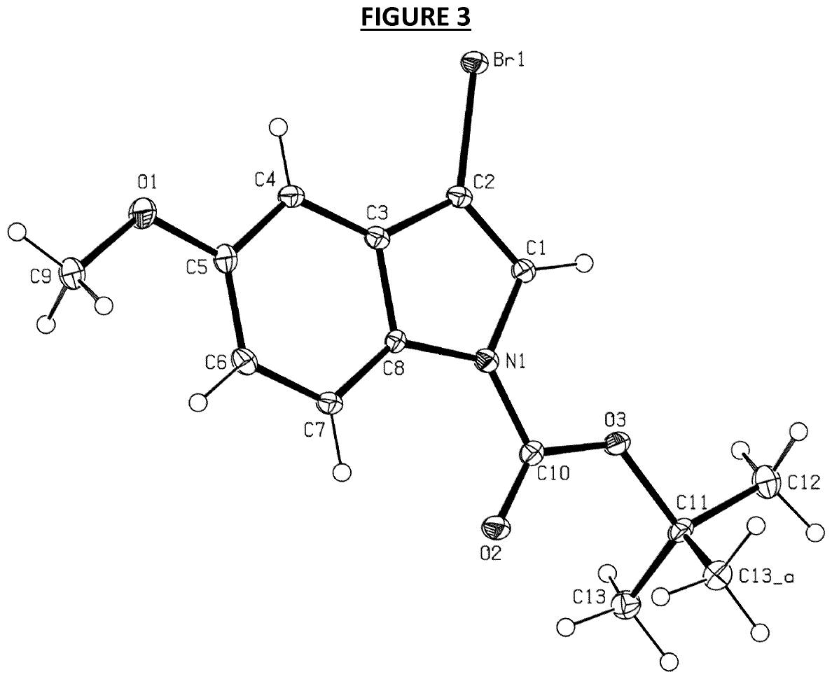

FIG. 3 shows the X-ray crystal structure of tert-butyl 3-bromo-5-methoxy-1H-indole-1-carboxylate.

### Example 17. Preparation of tert-butyl 1H-indole-1-carboxylate

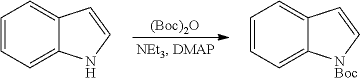 [SMILES: "(Boc)2O" ; c1ccc2[nH]ccc2c1 ; CC(C)(C)OC(=O)n1ccc2ccccc21]

A solution of (Boc)₂O(13.94 g, 64 mmol) in dichloromethane (20 ml) was added to a mixture of indole (6.8 g, 58 mmol), triethylamine (11.7 g, 116 mmol), DMAP (5 mg) in dichloromethane (70 ml) and the reaction stirred overnight at room temperature. It was quenched with saturated NaHCO₃ solution (50 ml) and the phases separated. The aqueous layer was extracted with dichloromethane (2×20 ml). The combined organic portion was washed with brine and dried over MgSO₄. The solvent was removed, and the residue eluted through a silica gel pad using ethyl acetate/hexanes (1:3) as eluent. The solvent was removed, and the residue was dried under vacuum to give the product as a pale-yellow oil. Yield=12.6 g.

### Example 18. Preparation of tert-butyl 3-bromo-1H-indole-1-carboxylate

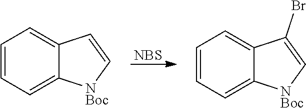

NBS (10.36 g, 58 mmol) was added to a mixture of tert-butyl 1H-indole-1-carboxylate (12.05 g, 55.4 mmol) and NH₄Cl (30 mg) in dichloromethane (150 ml) and the reaction stirred overnight at room temperature. Water (40 ml) was added, and the phases separated. The combined organic portion was washed with brine (30 ml), then water (20 ml), then dried over MgSO₄ and filtered. The solvent was removed, and the residue was eluted through a silica gel pad using EA/CH₂Cl₂/hexanes (1:2:10) as eluent. The solvent was removed, and the residue dried under vacuum to give the product as a white crystalline solid. Yield=16.2 g.

### Example 19. Preparation of 5-methoxy-1-tosyl-1H-indole

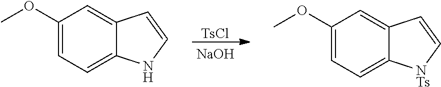

A solution of TsCl (2.6 g, 13.6 mmol) in toluene (20 ml) was added dropwise to a mixture of 5-methoxyindole (2.0 g, 13.6 mmol), 50% NaOH solution (14 ml) and TBAF (0.355 g, 1.36 mmol) with vigorous stirring at room temperature. Stirring was continued for another 3 hours after the addition was completed. The reaction was quenched with saturated NaHCO₃ solution (20 ml) and the phases separated. The aqueous layer was extracted with toluene (2×10 ml). The combined organic portion was washed with water and dried over MgSO₄. The solvent was removed, and the residue eluted through a silica gel pad using ethyl acetate as eluent. The solvent was removed, and the residue was dried under vacuum to give the product as a white solid. Yield=4.1 g.

### Example 20. Preparation of 3-bromo-5-methoxy-1-tosyl-1H-indole

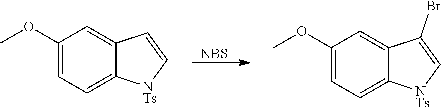

A solution of NBS (0.30 g, 1.7 mmol) in dichloromethane (5 ml) was added dropwise to a mixture of 5-methoxy-1-tosyl-1H-indole (0.50 g, 1.66 mmol) in dichloromethane (30 ml) at 0° C. The reaction was stirred overnight at room temperature. Water (40 ml) was added, and the phases separated. The combined organic portion was washed with brine (30 ml), then water (20 ml), then dried over MgSO₄ and filtered. The solvent was removed, and the residue was eluted through a silica gel pad using EA/CH₂Cl₂/hexanes (1:2:10) as eluent. The solvent was removed, and the residue dried under vacuum to give the product as a white crystalline solid. Yield=0.61 g.

### Example 21. Preparation of 3-iodo-4-methoxy-1H-indole

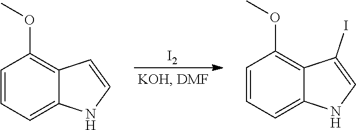

A solution of iodine (2.57 g, 10.1 mmol) in DMF (15 ml) was added dropwise to a mixture of 4-methoxyindole (1.5 g, 10.2 mmol) in DMF (15 ml) and KOH (1.66 g, 25 mmol) at room temperature. The mixture was stirred for 50 minutes, then the reaction mixture poured into ice-water (200 ml) containing 1% NH₄OH and 0.2% sodium sulphite. The precipitate was filtered, washed with ice-water and dried under vacuum. The product was obtained as a brown solid. Yield=2.55 g.

### Example 22. Preparation of tert-butyl 3-lodo-4-methoxy-1H-indole-1-carboxylate

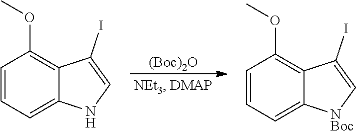 [SMILES: "(Boc)2O" ; COc1cccc2[nH]cc(I)c12 ; COc1cccc2c1c(I)cn2C(=O)OC(C)(C)C]

A solution of (Boc)₂O(2.24 g, 10.3 mmol) in dichloromethane (10 ml) was added to a mixture of 3-iodo-4-methoxy-1H-indole (2.55 g, 9.3 mmol), triethylamine (1.9 g, 18.6 mmol), DMAP (11 mg) in dichloromethane (30 ml) and the reaction stirred overnight at room temperature. It was quenched with saturated NaHCO₃ solution (20 ml) and the phases separated. The aqueous layer was extracted with dichloromethane (2×20 ml). The combined organic portion was washed with brine and dried over MgSO₄. The solvent was removed, and the residue eluted through a silica gel pad using CH₂Cl₂/hexanes (1:2) as eluent. The solvent was removed, and the residue was dried under vacuum to give the product as a grey crystalline solid, that darkens over time. It was stored in the dark. Yield=3.4 g.

### Example 23. Preparation of tert-butyl 3-bromo-5-(methoxy-d₃)-1H-indole-1-carboxylate

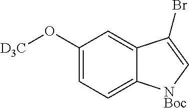 [SMILES: [2H]C([2H])([2H])Oc1ccc2c(c1)c(Br)cn2C(=O)OC(C)(C)C]

This was prepared from 5-(methoxy-d₃)-1H-indole using the procedures described in Examples 15 and 16.

### Example 24. Preparation of tert-butyl 3-bromo-4-(methoxy-d₃)-1H-indole-1-carboxylate

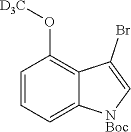 [SMILES: [2H]C([2H])([2H])Oc1cccc2c1c(Br)cn2C(=O)OC(C)(C)C]

This was prepared using 4-(methoxy-d₃)-1H-indole and the procedures described in Examples 7 and 8.

### Example 25. Preparation of tert-butyl 3-bromo-5-(methoxy-¹³C)-1H-indole-1-carboxylate

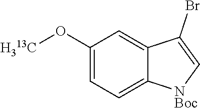 [SMILES: CC(C)(C)OC(=O)n1cc(Br)c2cc(O[13CH3])ccc21]

This was prepared from 5-(methoxy-¹³C)-1H-indole using the procedures described in Examples 15 and 16.

### Example 26. Preparation of tert-butyl 3-bromo-4-(methoxy-¹³C)-1H-indole-1-carboxylate

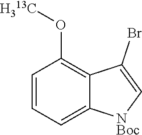 [SMILES: CC(C)(C)OC(=O)n1cc(Br)c2c(O[13CH3])cccc21]

This was prepared from 4-(methoxy-¹³C)-1H-indole using the procedures described in Examples 15 and 16.

### Example 27. General Procedure for the Preparation of α-Bromo Amides

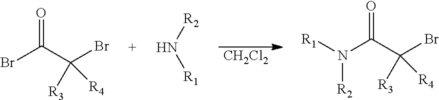

A solution of the amine (99.1 mmol) in dichloromethane (100 ml) was added to a solution of 2-bromoacetyl bromide (49.5 mmol) in dichloromethane (50 ml) at −16° C. over 30 minutes and the reaction mixture stirred for another 30 minutes after the addition was completed. It was allowed to warm to room temperature and stirred for another one hour. Water (50 ml) was added, and the phases separated. The aqueous layer was extracted with dichloromethane (2×10 ml). The combined organic portion was washed with brine, dried over MgSO₄, filtered and the solvent removed under vacuum to yield the product.

This procedure was used for the preparation of the α-bromo amides below.

### Example 27(i). 2-Bromo-N,N-dimethylacetamide

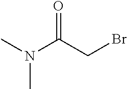

### Example 27(ii). 2-Bromo-N,N-dimethylpropanamide

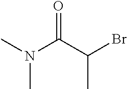

### Example 27(iii). 2-Bromo-N,N-dimethylbutanamide

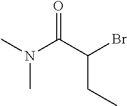

### Example 27(iv). 2-Bromo-N,N-diethylacetamide

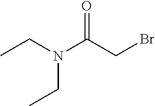

### Example 27(v). 2-Bromo-N,N-diisopropylacetamide

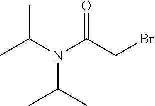

### Example 27(vi). 2-Bromo-1-(pyrrolidin-1-yl)ethanone

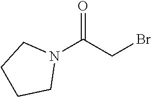

### Example 27(vii). 2-Bromo-1-(piperidin-1-yl)ethanone

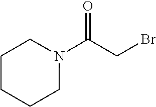

### Example 27(viii). 2-Bromo-1-morpholinoethanone

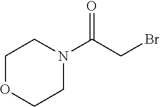

### Example 27(ix). N,N-Dibenzyl-2-bromoacetamide

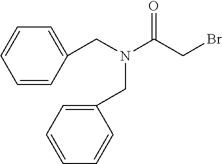 [SMILES: O=C(CBr)N(Cc1ccccc1)Cc1ccccc1]

### Example 27(x). N-Benzyl-2-bromo-N-methylacetamide

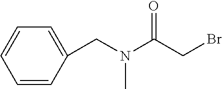

### Example 27(xi). 2-(2-Bromoacetyl)isoindoline-1,3-dione

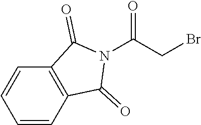 [SMILES: O=C(CBr)N1C(=O)c2ccccc2C1=O]

### Example 27(xii). 2-Bromo-N,N-bis(methyl-d₃)acetamide

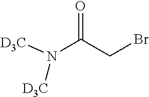 [SMILES: [2H]C([2H])([2H])N(C(=O)CBr)C([2H])([2H])[2H]]

### Example 27(xiii). 2-Bromo-N,N-di(methyl-¹³C)acetamide

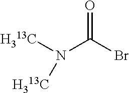

### Example 27(xiv). 2-Bromo-1-(morpholino-d₈)ethan-1-one

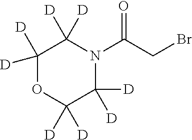 [SMILES: [2H]C1([2H])OC([2H])([2H])C([2H])([2H])N(C(=O)CBr)C1([2H])[2H]]

### 3-Bromo-1-methylpyrrolidin-2-one was obtained from commercial sources

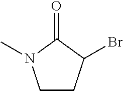

### Example 28. General procedure for the preparation of zinc amide enolates

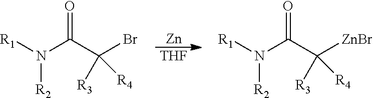

Zinc granules (0.90 g, 13.76 mmol) were dried under vacuum while heating in a Schlenk flask, then refilled with argon. The flask was cooled to room temperature and a pinch of iodine was added while the flask was still warm. The α-bromo amide (12.53 mmol) was degassed with argon and dry THE (22 ml) added. The amide solution was added dropwise to the zinc at 0° C. with vigorous stirring. The mixture was allowed to warm to room temperature after the addition was completed and the stirring continued until all the amide reacted. The zinc amide enolates were used as a suspension in THF.

This procedure was used for the preparation of the zinc amide enolates below.

### Example 28(i). (2-(Dimethylamino)-2-oxoethyl)zinc(II) bromide

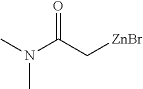

### Example 28(ii). (1-(Dimethylamino)-1-oxopropan-2-yl)zinc(II) bromide

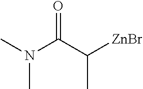

### Example 28(iii). (1-(Dimethylamino)-1-oxobutan-2-yl)zinc(II) bromide

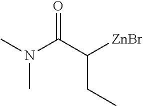

### Example 28(iv). (2-(Diethylamino)-2-oxoethyl)zinc(II) bromide

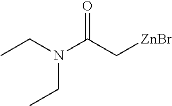

### Example 28(v). (2-(Diisopropylamino)-2-oxoethyl)zinc(II) bromide

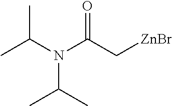

### Example 28(vi). (2-Oxo-2-(pyrrolidin-1-yl)ethyl)zinc(II) bromide

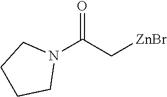

### Example 28(vii). (2-Oxo-2-(piperidin-1-yl)ethyl)zinc(II) bromide

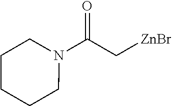

### Example 28(viii). (2-Morpholino-2-oxoethyl)zinc(II) bromide

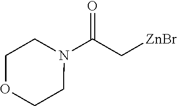

### Example 28(ix). (2-(Dibenzylamino)-2-oxoethyl)zinc(II) bromide

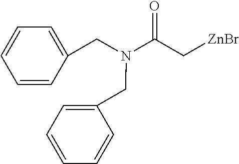

### Example 28(x). (2-(Benzyl(methyl)amino)-2-oxoethyl)zinc(II) bromide

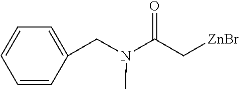

### Example 28(xi). (2-(1,3-Dioxoisoindolin-2-yl)-2-oxoethyl)zinc(II) bromide

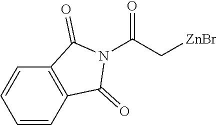

### Example 28(xii). (2-bis(methyl-d₃)amino)-2-oxoethyl)zinc(II) bromide

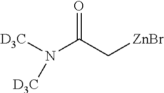

### Example 28(xiii). (2-(di(methyl-¹³C)amino)-2-oxoethyl)zinc(II) bromide

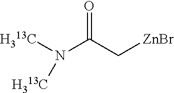

### Example 28(xiv). (2-(Morpholino-d₈)-2-oxoethyl)zinc(II) bromide

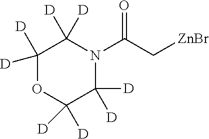

### Example 28(xv). (1-Methyl-2-oxopyrrolidin-3-yl)zinc(II) bromide

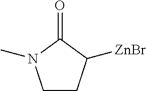

### Example 29. Catalyst screening for the Negishi coupling of tert-butyl 3-bromo-5-methoxy-1H-indole-1-carboxylate and (2-(dimethylamino)-2-oxoethyl)zinc(II) bromide

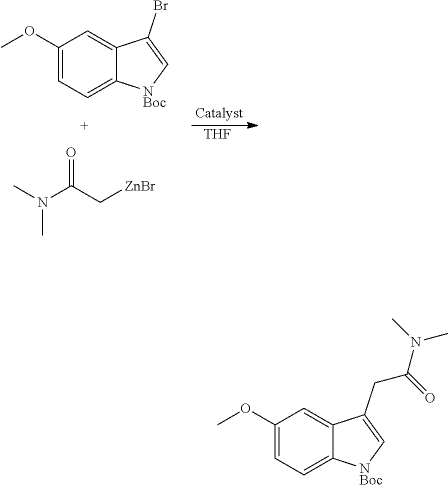

A THE suspension of (2-(dimethylamino)-2-oxoethyl)zinc(II) bromide (1.0 ml, 0.5 mmol) was added to a mixture of tert-butyl 3-bromo-5-methoxy-1H-indole-1-carboxylate (100 mg, 0.3 mmol) and the catalyst (0.015 mmol) in a Schlenk flask under argon. The mixture was stirred at the required temperature under argon and the reaction progress monitored by TLC and ¹H NMR. The results for the various catalyst investigated are summarized in Table 1.

TABLE 1
Catalysts used in Example 29.

| Example | Catalyst | Temp./° C. | Time/hour | Conv./% |
|---|---|---|---|---|
| i | PdCl₂(dppf) | 65 | 20 | <5 |
| ii | PdCl₂(dppe) | 65 | 20 | 0 |
| iii | NiCl₂(dppf) | 65 | 20 | 0 |
| iv | NiCl₂(dppe) | 65 | 20 | 0 |
| vi | PdCl₂(PPh₃)₂ | 65 | 20 | 0 |
| vii | PdCl₂(Xantphos) | 65 | 20 | <5 |
| viii | (Xantphos)PdG2 | 65 | 20 | <5 |
| ix | XPhosPdG1 | 65 | 20 | 10 |
| x | RuPhosPdG1 | 65 | 20 | 10 |
| xi | SPhosPdG1 | 65 | 20 | 10 |
| xii | tBuXPhosPdG1 | 65 | 20 | 100 |
| xiii | tBuXPhosPdG1 | 35 | 20 | 25 |
| xiv | tBuXPhosPdG1 | 50 | 20 | 50 |
| xv | tBuXPhosPdG1 | 65 | 5 | 75 |
| xvi | tBuXPhosPdG1 | 65 | 16 | 100 |
| xvii | QPhos/Pd(dba)₂ | 65 | 20 | <5 |
| xviii | PtBu₃PdG2 | 65 | 20 | <5 |
| xix | PCy₃PdG2 | 65 | 20 | <5 |
| xx | BrettPhosPdG1 | 65 | 20 | 10 |
| xxi | PEPPSI-IPr | 65 | 20 | 0 |
| xxii | PEPPSI-SIPr | 65 | 20 | 0 |

### Example 30. General Procedure for the Negishi Coupling of 3-Halo-Indoles and Zinc Amide Enolates

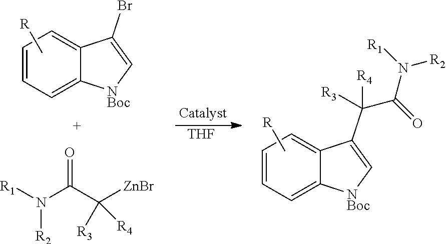

A suspension of the zinc amide enolate (2.5 mmol) was added to a mixture of the 3-halo-indole (1.5 mmol) and the catalyst tBuXPhosPdG1 (50 mg, 0.073 mmol) in a Schlenk flask under argon. The mixture was stirred at 65° C. under argon for 16 hours. it was cooled to room temperature and the solvent removed under reduced pressure. Water (10 ml) and ether (10 ml) were added with stirring and the phases separated. The ether layer was dried over MgSO₄, then filtered and the solvent removed under reduced pressure. The residue was eluted through a silica gel pad. The eluent was evaporated to yield the crude product, which was purified by silica gel chromatography.

This procedure was used for the preparation of the products below.

### Example 30(i). tert-Butyl 3-(2-(dimethylamino)-2-oxoethyl)-5-methoxy-1H-indole-1-carboxylate

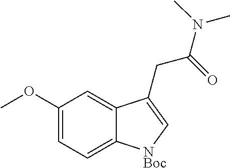

### Example 30(ii). tert-Butyl 3-(1-(dimethylamino)-1-oxopropan-2-yl)-5-methoxy-1H-indole-1-carboxylate

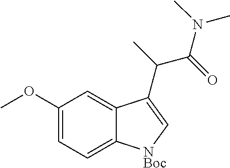

### Example 30(iii). tert-Butyl 3-(1-(dimethylamino)-1-oxobutan-2-yl)-5-methoxy-1H-indole-1-carboxylate

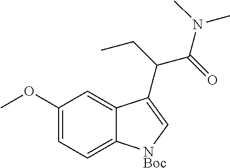

### Example 30(iv). tert-Butyl 5-methoxy-3-(2-oxo-2-(pyrrolidin-1-yl)ethyl)-1H-indole-1-carboxylate

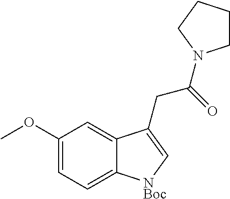 [SMILES: COc1ccc2c(c1)c(CC(=O)N1CCCC1)cn2C(=O)OC(C)(C)C]

### Example 30(v). tert-Butyl 5-methoxy-3-(2-morpholino-2-oxoethyl)-1H-indole-1-carboxylate

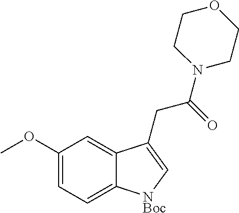 [SMILES: COc1ccc2c(c1)c(CC(=O)N1CCOCC1)cn2C(=O)OC(C)(C)C]

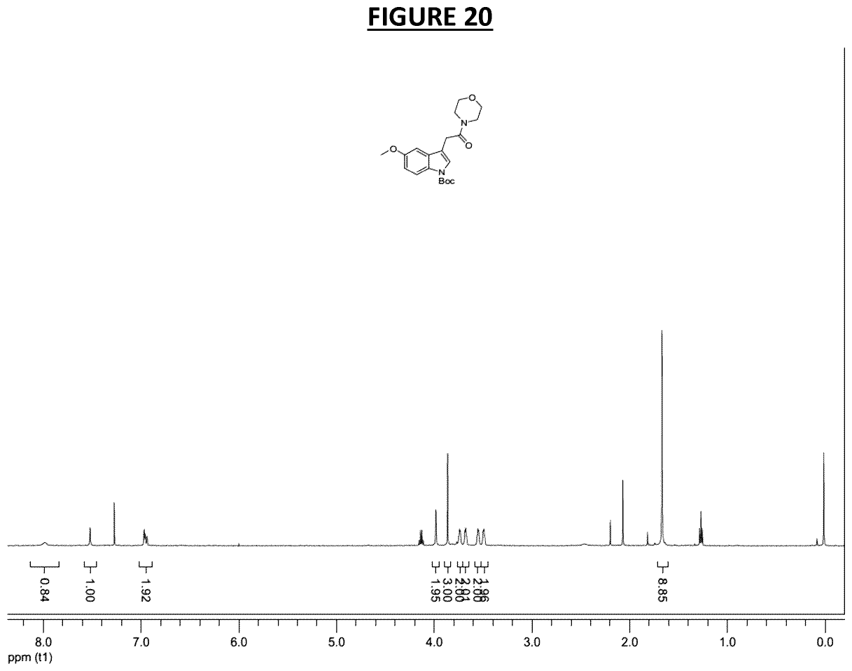

FIG. 20 shows the ¹H NMR spectrum of tert-Butyl 5-methoxy-3-(2-morpholino-2-oxoethyl)-1H-indole-1-carboxylate.

### Example 30(vi). tert-Butyl 3-(2-(diethylamino)-2-oxoethyl)-5-methoxy-1H-indole-1-carboxylate

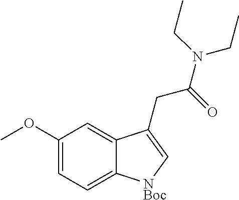 [SMILES: CCN(CC)C(=O)Cc1cn(C(=O)OC(C)(C)C)c2ccc(OC)cc12]

### Example 30(vii). tert-Butyl 3-(2-(diisopropylamino)-2-oxoethyl)-5-methoxy-1H-indole-1-carboxylate

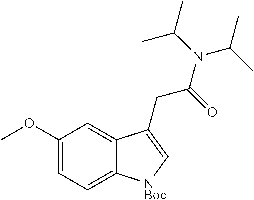 [SMILES: COc1ccc2c(c1)c(CC(=O)N(C(C)C)C(C)C)cn2C(=O)OC(C)(C)C]

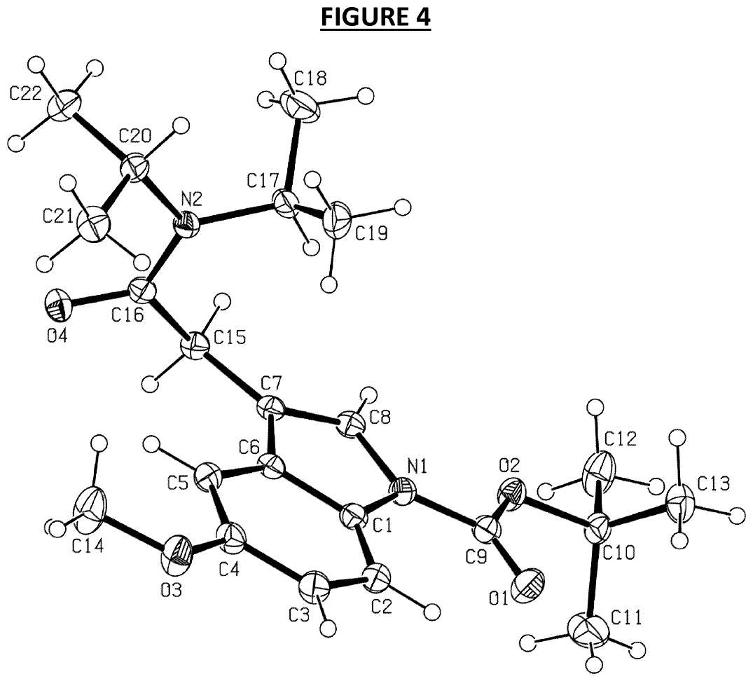

FIG. 4 shows the X-ray crystal structure of tert-Butyl 3-(2-(diisopropylamino)-2-oxoethyl)-5-methoxy-1H-indole-1-carboxylate.

### Example 30(viii). tert-Butyl 3-(2-(benzyl(methyl)amino)-2-oxoethyl)-5-methoxy-1H-indole-1-carboxylate

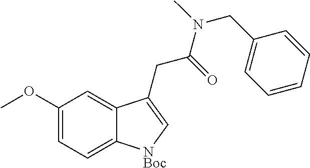 [SMILES: COc1ccc2c(c1)c(CC(=O)N(C)Cc1ccccc1)cn2C(=O)OC(C)(C)C]

### Example 30(ix). tert-Butyl 3-(2-(dibenzylamino)-2-oxoethyl)-5-methoxy-1H-indole-1-carboxylate

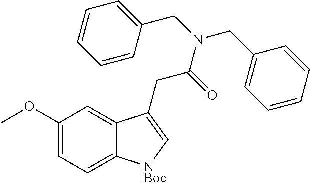 [SMILES: COc1ccc2c(c1)c(CC(=O)N(Cc1ccccc1)Cc1ccccc1)cn2C(=O)OC(C)(C)C]

### Example 30(x). tert-Butyl 3-(2-(bis(methyl-d₃)amino)-2-oxoethyl)-5-methoxy-1H-indole-1-carboxylate

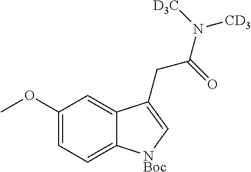 [SMILES: [2H]C([2H])([2H])N(C(=O)Cc1cn(C(=O)OC(C)(C)C)c2ccc(OC)cc12)C([2H])([2H])[2H]]

### Example 30(xi). tert-Butyl 3-(2-(di(methyl-¹³C)amino)-2-oxoethyl)-5-methoxy-1H-indole-1-carboxylate

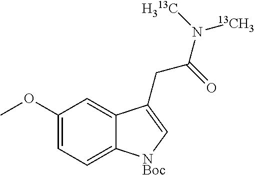

### Example 30(xii). tert-Butyl 5-methoxy-3-(2-(morpholino-d₈)-2-oxoethyl)-1H-indole-1-carboxylate

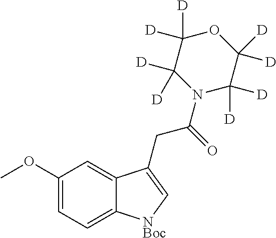 [SMILES: [2H]C1([2H])OC([2H])([2H])C([2H])([2H])N(C(=O)Cc2cn(C(=O)OC(C)(C)C)c3ccc(OC)cc23)C1([2H])[2H]]

### Example 30(xiii). tert-Butyl 3-(2-(dimethylamino)-2-oxoethyl)-1H-indole-1-carboxylate

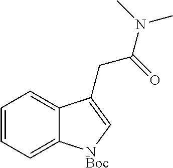 [SMILES: CN(C)C(=O)Cc1cn(C(=O)OC(C)(C)C)c2ccccc12]

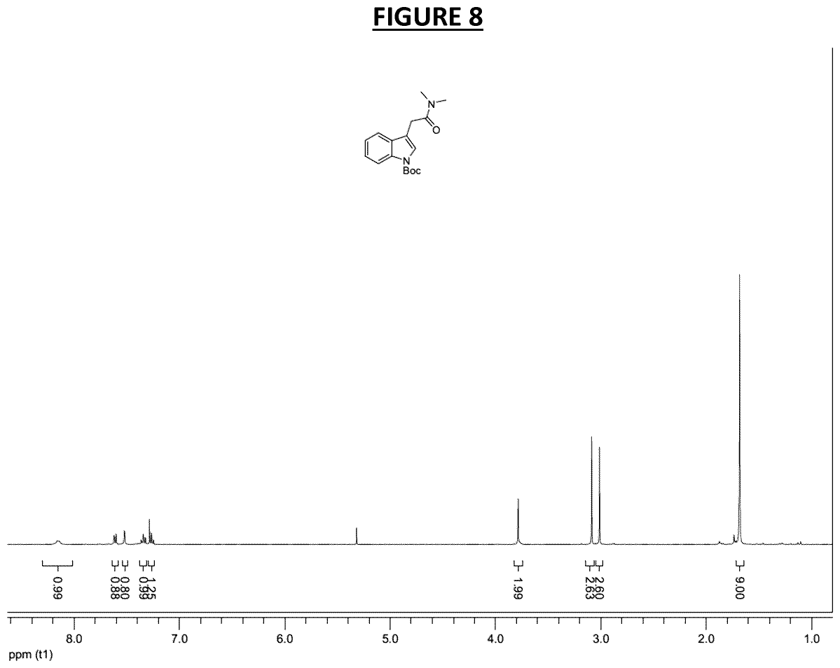

FIG. 8 shows the ¹H NMR spectrum of tert-Butyl 3-(2-(dimethylamino)-2-oxoethyl)-1H-indole-1-carboxylate.

### Example 30(xiv). tert-Butyl 3-(1-(dimethylamino)-1-oxopropan-2-yl)-1H-indole-1-carboxylate

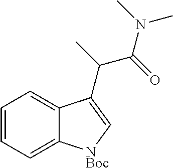 [SMILES: CC(C(=O)N(C)C)c1cn(C(=O)OC(C)(C)C)c2ccccc12]

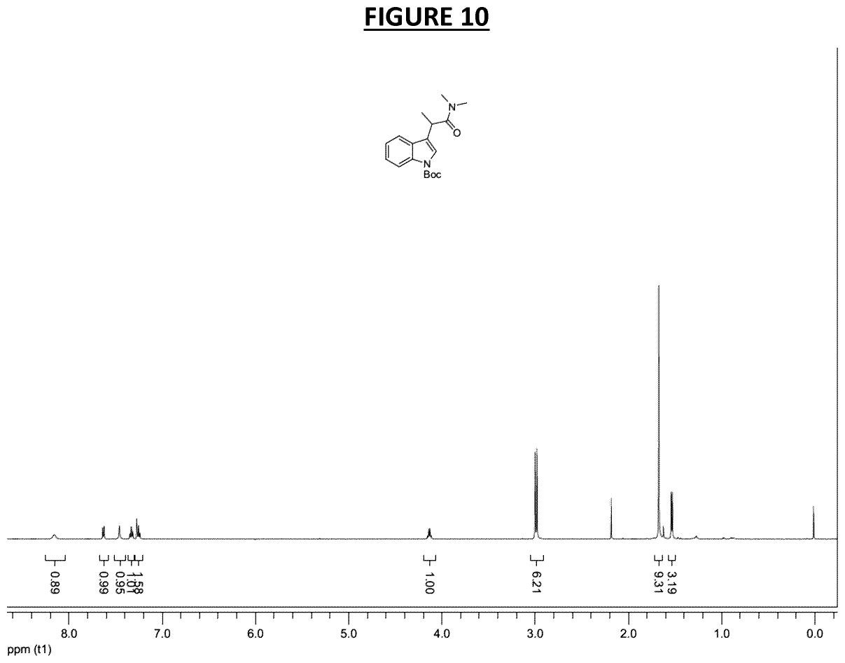

FIG. 10 shows the ¹H NMR spectrum of tert-Butyl 3-(1-(dimethylamino)-1-oxopropan-2-yl)-1H-indole-1-carboxylate.

### Example 30(xv). tert-Butyl 3-(1-(dimethylamino)-1-oxobutan-2-yl)-1H-indole-1-carboxylate

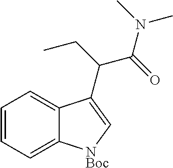 [SMILES: CCC(C(=O)N(C)C)c1cn(C(=O)OC(C)(C)C)c2ccccc12]

### Example 30(xvi). tert-Butyl 3-(2-oxo-2-(pyrrolidin-1-yl)ethyl)-1H-indole-1-carboxylate

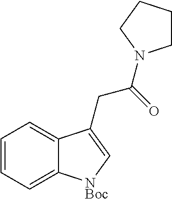 [SMILES: CC(C)(C)OC(=O)n1cc(CC(=O)N2CCCC2)c2ccccc21]

### Example 30(xvii). tert-Butyl 3-(2-morpholino-2-oxoethyl)-1H-indole-1-carboxylate

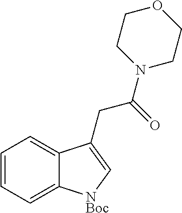 [SMILES: CC(C)(C)OC(=O)n1cc(CC(=O)N2CCOCC2)c2ccccc21]

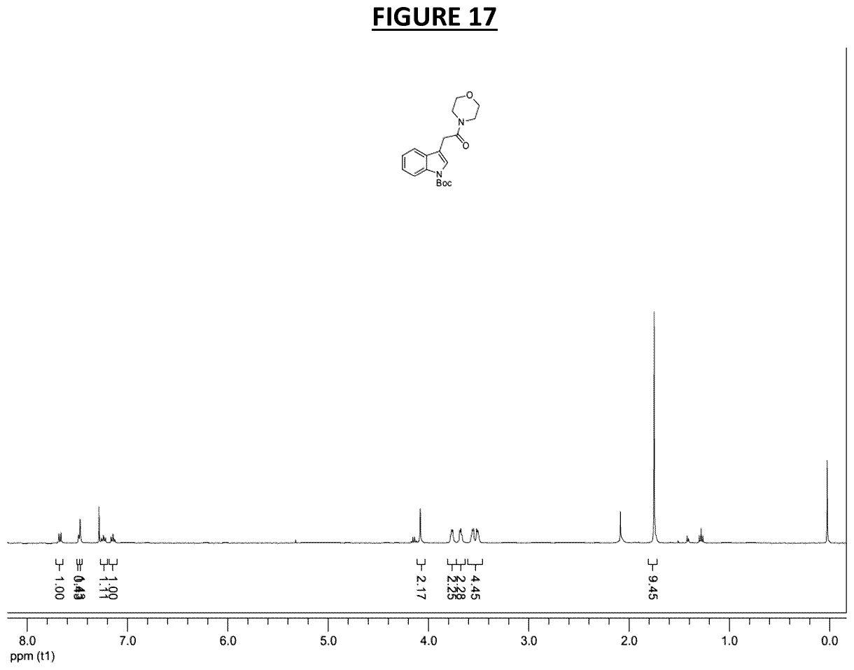

FIG. 17 shows the ¹H NMR spectrum of tert-Butyl 3-(2-morpholino-2-oxoethyl)-1H-indole-1-carboxylate.

### Example 30(xviii). tert-Butyl 3-(2-(diethylamino)-2-oxoethyl)-1H-indole-1-carboxylate

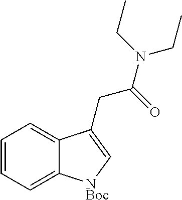 [SMILES: CCN(CC)C(=O)Cc1cn(C(=O)OC(C)(C)C)c2ccccc12]

### Example 30(xix). tert-Butyl 3-(2-(diisopropylamino)-2-oxoethyl)-1H-indole-1-carboxylate

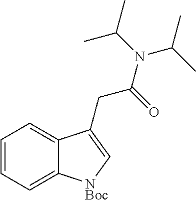 [SMILES: CC(C)N(C(=O)Cc1cn(C(=O)OC(C)(C)C)c2ccccc12)C(C)C]

### Example 30(xx). tert-Butyl 3-(2-(benzyl(methyl)amino)-2-oxoethyl)-1H-indole-1-carboxylate

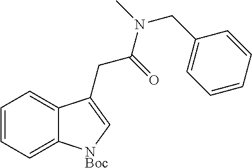 [SMILES: CN(Cc1ccccc1)C(=O)Cc1cn(C(=O)OC(C)(C)C)c2ccccc12]

### Example 30(xxi). tert-Butyl 3-(2-(dibenzylamino)-2-oxoethyl)-1H-indole-1-carboxylate

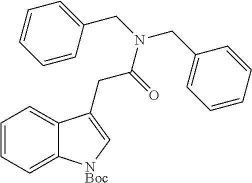 [SMILES: CC(C)(C)OC(=O)n1cc(CC(=O)N(Cc2ccccc2)Cc2ccccc2)c2ccccc21]

### Example 30(xxii). tert-Butyl 3-(2-(bis(methyl-d₃)amino)-2-oxoethyl)-1H-indole-1-carboxylate

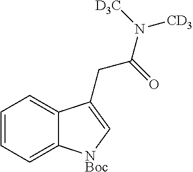 [SMILES: [2H]C([2H])([2H])N(C(=O)Cc1cn(C(=O)OC(C)(C)C)c2ccccc12)C([2H])([2H])[2H]]

### Example 30(xxiii). tert-Butyl 3-(2-(di(methyl-¹³C)amino)-2-oxoethyl)-1H-indole-1-carboxylate

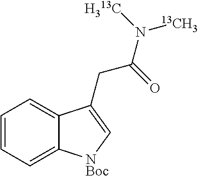 [SMILES: CC(C)(C)OC(=O)n1cc(CC(=O)N([13CH3])[13CH3])c2ccccc21]

### Example 30(xxiv). tert-Butyl 3-(2-(morpholino-d₈)-2-oxoethyl)-1H-indole-1-carboxylate

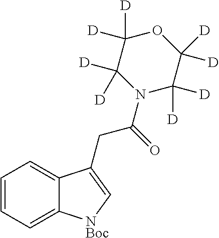 [SMILES: [2H]C1([2H])OC([2H])([2H])C([2H])([2H])N(C(=O)Cc2cn(C(=O)OC(C)(C)C)c3ccccc23)C1([2H])[2H]]

### Example 30(xxv). tert-Butyl 3-(2-(dimethylamino)-2-oxoethyl)-4-methoxy-1H-indole-1-carboxylate

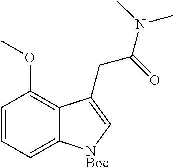 [SMILES: COc1cccc2c1c(CC(=O)N(C)C)cn2C(=O)OC(C)(C)C]

### Example 30(xxvi). tert-Butyl 3-(1-(dimethylamino)-1-oxopropan-2-yl)-4-methoxy-1H-indole-1-carboxylate

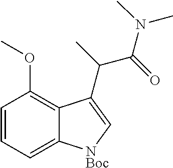 [SMILES: COc1cccc2c1c(C(C)C(=O)N(C)C)cn2C(=O)OC(C)(C)C]

### Example 30(xxvii). tert-Butyl 3-(1-(dimethylamino)-1-oxobutan-2-yl)-4-methoxy-1H-indole-1-carboxylate

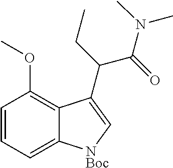 [SMILES: CCC(C(=O)N(C)C)c1cn(C(=O)OC(C)(C)C)c2cccc(OC)c12]

### Example 30(xxviii). tert-Butyl 4-methoxy-3-(2-oxo-2-(pyrrolidin-1-yl)ethyl)-1H-indole-1-carboxylate

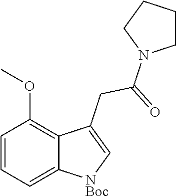 [SMILES: COc1cccc2c1c(CC(=O)N1CCCC1)cn2C(=O)OC(C)(C)C]

### Example 30(xxix). tert-Butyl 4-methoxy-3-(2-morpholino-2-oxoethyl)-1H-indole-1-carboxylate

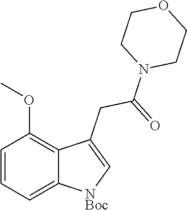 [SMILES: COc1cccc2c1c(CC(=O)N1CCOCC1)cn2C(=O)OC(C)(C)C]

### Example 30(xxx). tert-Butyl 3-(2-(diethylamino)-2-oxoethyl)-4-methoxy-1H-indole-1-carboxylate

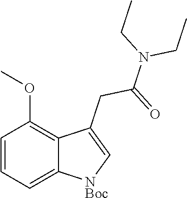 [SMILES: CCN(CC)C(=O)Cc1cn(C(=O)OC(C)(C)C)c2cccc(OC)c12]

### Example 30(xxxi). tert-Butyl 3-(2-(diisopropylamino)-2-oxoethyl)-4-methoxy-1H-indole-1-carboxylate

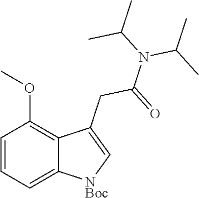 [SMILES: COc1cccc2c1c(CC(=O)N(C(C)C)C(C)C)cn2C(=O)OC(C)(C)C]

### Example 30(xxxii). tert-Butyl 3-(2-(benzyl(methyl)amino)-2-oxoethyl)-4-methoxy-1H-indole-1-carboxylate

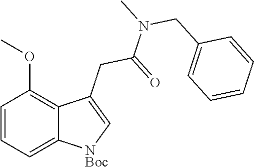 [SMILES: COc1cccc2c1c(CC(=O)N(C)Cc1ccccc1)cn2C(=O)OC(C)(C)C]

### Example 30(xxxiii). tert-Butyl 3-(2-(dibenzylamino)-2-oxoethyl)-4-methoxy-1H-indole-1-carboxylate

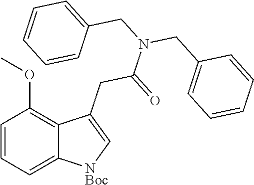 [SMILES: COc1cccc2c1c(CC(=O)N(Cc1ccccc1)Cc1ccccc1)cn2C(=O)OC(C)(C)C]

### Example 30(xxxiv). tert-Butyl 3-(2-(bis(methyl-d₃)amino)-2-oxoethyl)-4-methoxy-1H-indole-1-carboxylate

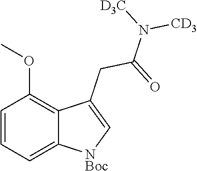 [SMILES: [2H]C([2H])([2H])N(C(=O)Cc1cn(C(=O)OC(C)(C)C)c2cccc(OC)c12)C([2H])([2H])[2H]]

### Example 30(xxxv). tert-Butyl 3-(2-(di(methyl-¹³C)amino)-2-oxoethyl)-4-methoxy-1H-indole-1-carboxylate

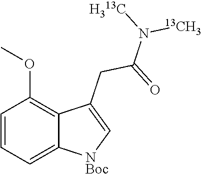 [SMILES: COc1cccc2c1c(CC(=O)N([13CH3])[13CH3])cn2C(=O)OC(C)(C)C]

### Example 30(xxxvi). tert-Butyl 4-methoxy-3-(2-(morpholino-d₈)-2-oxoethyl)-1H-indole-1-carboxylate

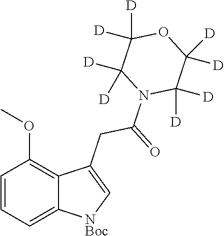 [SMILES: [2H]C1([2H])OC([2H])([2H])C([2H])([2H])N(C(=O)Cc2cn(C(=O)OC(C)(C)C)c3cccc(OC)c23)C1([2H])[2H]]

### Example 30(xxxvii). tert-Butyl 3-(2-(dimethylamino)-2-oxoethyl)-5-(methoxy-d₃)-1H-indole-1-carboxylate

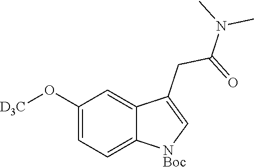 [SMILES: [2H]C([2H])([2H])Oc1ccc2c(c1)c(CC(=O)N(C)C)cn2C(=O)OC(C)(C)C]

### Example 30(xxxviii). tert-Butyl 3-(2-(dimethylamino)-2-oxoethyl)-4-(methoxy-d₃)-1H-indole-1-carboxylate

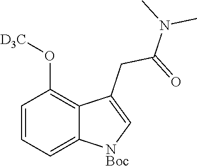 [SMILES: [2H]C([2H])([2H])Oc1cccc2c1c(CC(=O)N(C)C)cn2C(=O)OC(C)(C)C]

### Example 30(xxxix). tert-Butyl 5-acetoxy-3-(2-(dimethylamino)-2-oxoethyl)-1H-indole-1-carboxylate

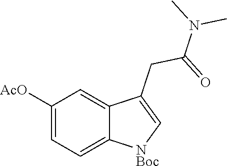 [SMILES: CC(=O)Oc1ccc2c(c1)c(CC(=O)N(C)C)cn2C(=O)OC(C)(C)C]

### Example 30(xxxx). tert-Butyl 4-acetoxy-3-(2-(dimethylamino)-2-oxoethyl)-1H-indole-1-carboxylate

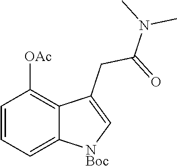 [SMILES: CC(=O)Oc1cccc2c1c(CC(=O)N(C)C)cn2C(=O)OC(C)(C)C]

### Example 30(xxxxi). tert-Butyl 5-methoxy-3-(1-methyl-2-oxopyrrolidin-3-yl)-1H-indole-1-carboxylate

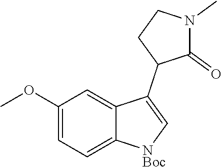

### Example 30(xxxxii). tert-Butyl 4-methoxy-3-(1-methyl-2-oxopyrrolidin-3-yl)-1H-indole-1-carboxylate

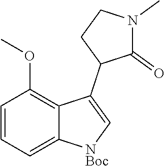 [SMILES: COc1cccc2c1c(C1CCN(C)C1=O)cn2C(=O)OC(C)(C)C]

### Example 30(xxxxiii). tert-Butyl 3-(1-methyl-2-oxopyrrolidin-3-yl)-1H-indole-1-carboxylate

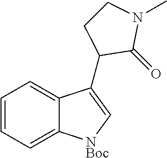 [SMILES: CN1CCC(c2cn(C(=O)OC(C)(C)C)c3ccccc23)C1=O]

### Example 31. General Procedure for the Preparation of 2-(1H-indol-3-yl) acetamides

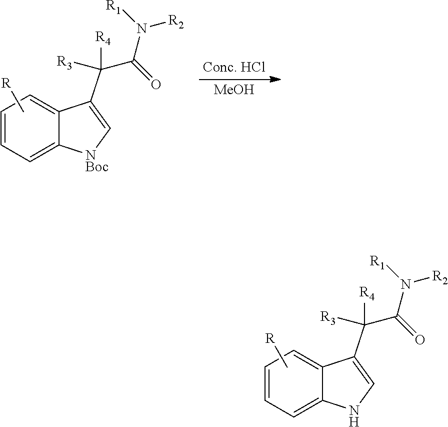

A mixture of Conc. HCl (1.0 ml) and methanol (2 ml) was added to the tert-butyl 3-(2-amino-2-oxoethyl)-1H-indole-1-carboxylate (50 mg) and the mixture stirred for 12-24 hours at room temperature until the reaction was completed (TLC). The mixture was evaporated under reduced pressure and sodium carbonate solution added to the residue. The mixture was stirred for 10 minutes, then dichloromethane added, and the phases separated. The organic layer was dried over MgSO₄, then filtered and the solvent removed under reduced pressure. The residue was eluted through a silica gel pad. The eluent was evaporated to yield the crude product, which was purified by silica gel chromatography.

This procedure was used for the preparation of the products below.

### Example 31(i). 2-(5-Methoxy-1H-indol-3-yl)-N,N-dimethylacetamide

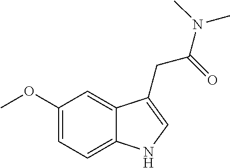

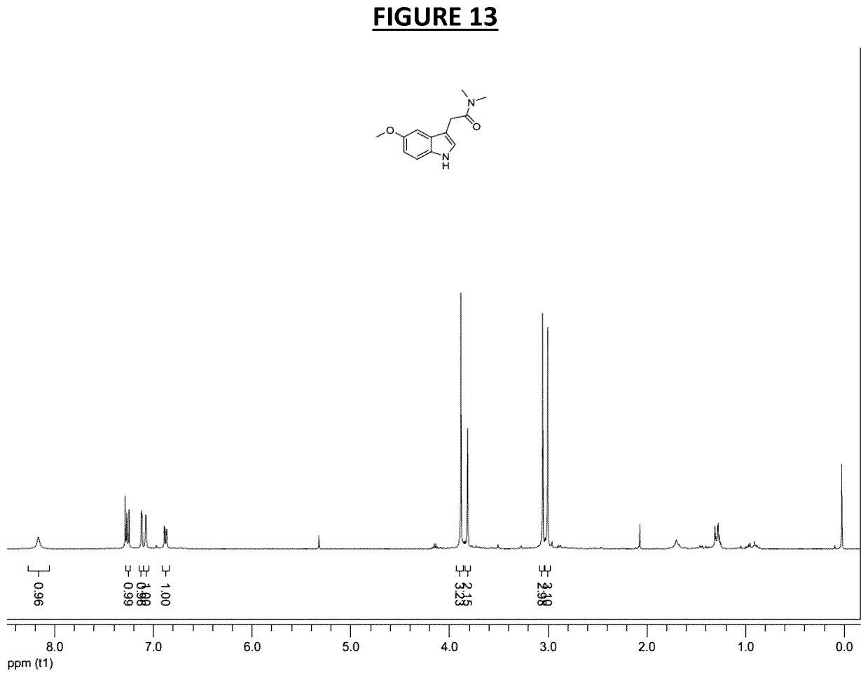

FIG. 13 shows the ¹H NMR spectrum of 2-(5-Methoxy-1H-indol-3-yl)-N,N-dimethylacetamide.

### Example 31(ii). 2-(5-Methoxy-1H-indol-3-yl)-N,N-dimethylpropanamide

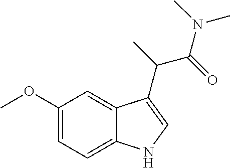

### Example 31(iii). 2-(5-Methoxy-1H-indol-3-yl)-N,N-dimethylbutanamide

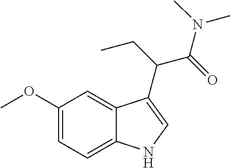

### Example 31(iv). 2-(5-Methoxy-1H-indol-3-yl)-1-(pyrrolidin-1-yl)ethanone

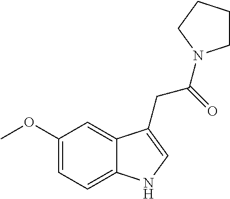 [SMILES: COc1ccc2[nH]cc(CC(=O)N3CCCC3)c2c1]

### Example 31(v). 2-(5-Methoxy-1H-indol-3-yl)-1-morpholinoethanone

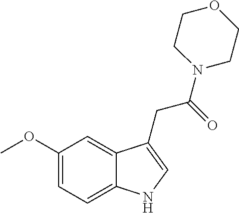 [SMILES: COc1ccc2[nH]cc(CC(=O)N3CCOCC3)c2c1]

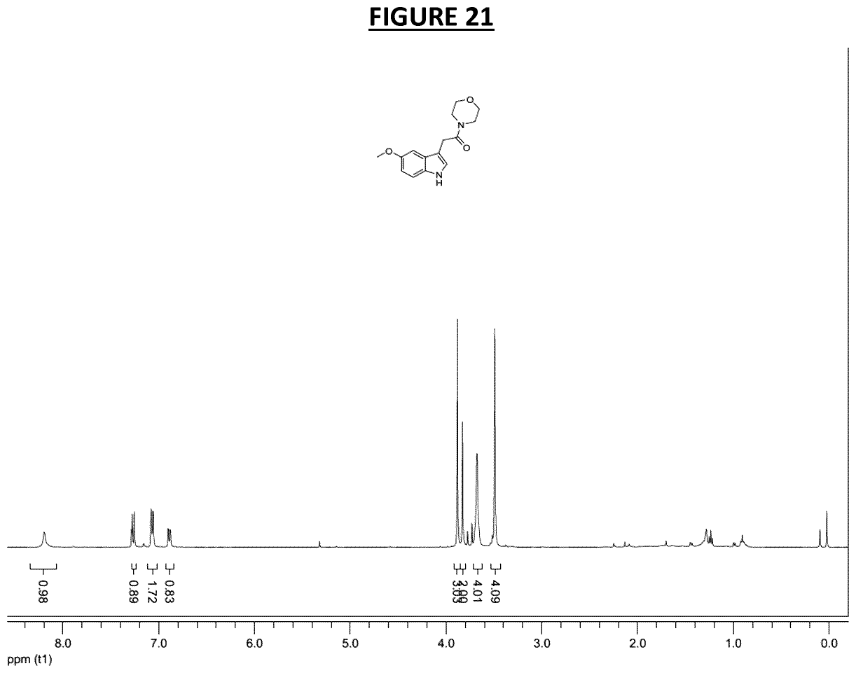

FIG. 21 shows the ¹H NMR spectrum of 2-(5-Methoxy-1H-indol-3-yl)-1-morpholinoethanone.

### Example 31(vi). N,N-diethyl-2-(5-methoxy-1H-indol-3-yl)acetamide

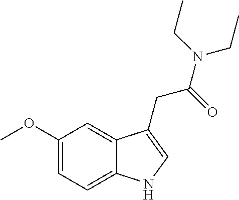 [SMILES: CCN(CC)C(=O)Cc1c[nH]c2ccc(OC)cc12]

### Example 31(vii). N,N-Diisopropyl-2-(5-methoxy-1H-indol-3-yl)acetamide

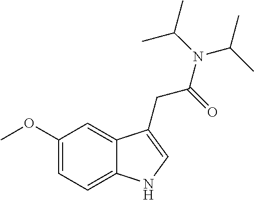 [SMILES: COc1ccc2[nH]cc(CC(=O)N(C(C)C)C(C)C)c2c1]

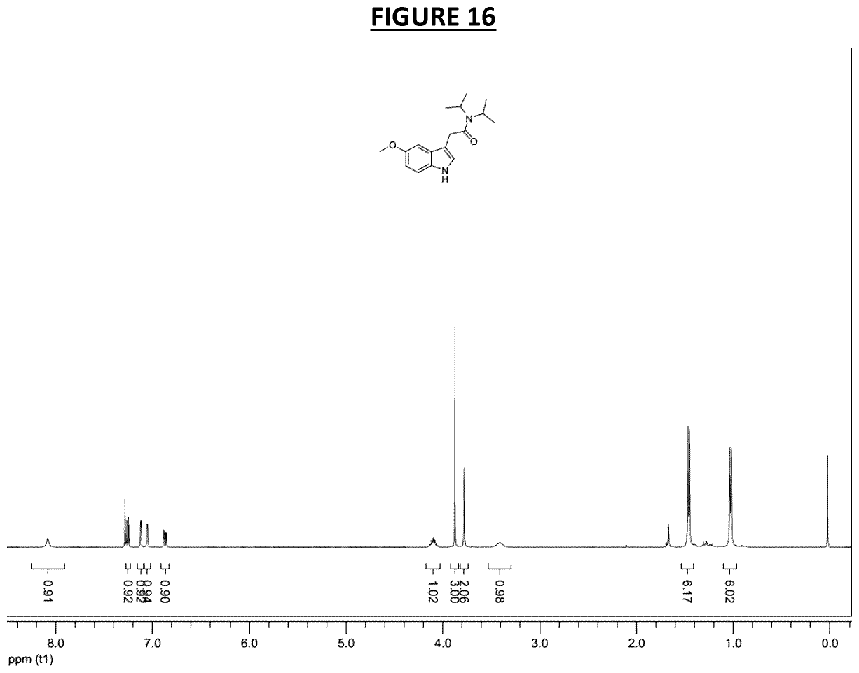

FIG. 16 shows the ¹H NMR spectrum of N,N-Diisopropyl-2-(5-methoxy-1H-indol-3-yl)acetamide.

### Example 31(viii). N-Benzyl-2-(5-methoxy-1H-indol-3-yl)-N-methylacetamide

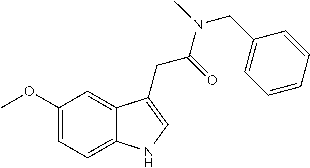 [SMILES: COc1ccc2[nH]cc(CC(=O)N(C)Cc3ccccc3)c2c1]

### Example 31(ix). N,N-dibenzyl-2-(5-methoxy-1H-indol-3-yl)acetamide

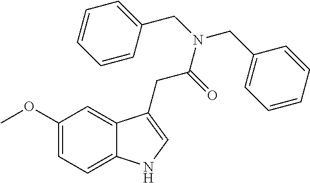 [SMILES: COc1ccc2[nH]cc(CC(=O)N(Cc3ccccc3)Cc3ccccc3)c2c1]

### Example 31(x). 2-(5-Methoxy-1H-indol-3-yl)-N,N-bis(dimethyl-d₃)acetamide

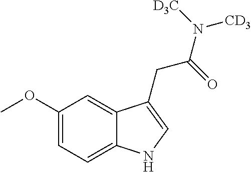 [SMILES: [2H]C([2H])([2H])N(C(=O)Cc1c[nH]c2ccc(OC)cc12)C([2H])([2H])[2H]]

### Example 31(xi). 2-(5-Methoxy-1H-indol-3-yl)-N,N-di(methyl-¹³C)acetamide

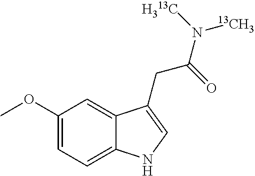

### Example 31(xii). 2-(5-Methoxy-1H-indol-3-yl)-1-(morpholino-d₈)ethanone

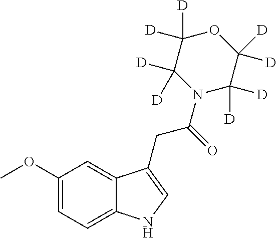 [SMILES: [2H]C1([2H])OC([2H])([2H])C([2H])([2H])N(C(=O)Cc2c[nH]c3ccc(OC)cc23)C1([2H])[2H]]

### Example 31(xiii). 2-(1H-indol-3-yl)-N,N-dimethylacetamide

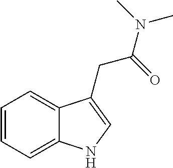

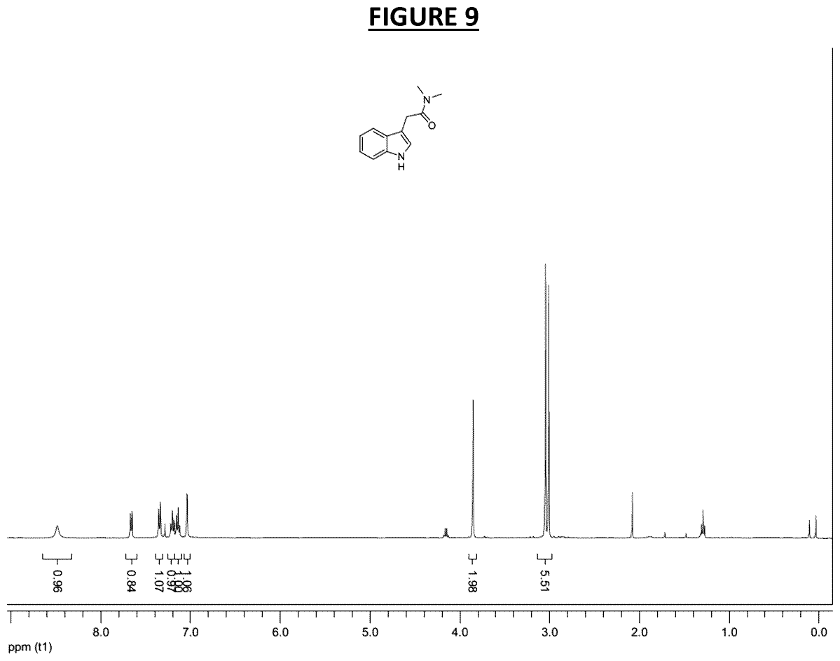

FIG. 9 shows the ¹H NMR spectrum of 2-(1H-indol-3-yl)-N,N-dimethylacetamide.

### Example 31(xiv). 2-(1H-indol-3-yl)-N,N-dimethylpropanamide

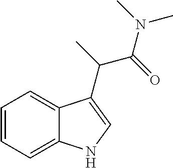 [SMILES: CC(C(=O)N(C)C)c1c[nH]c2ccccc12]

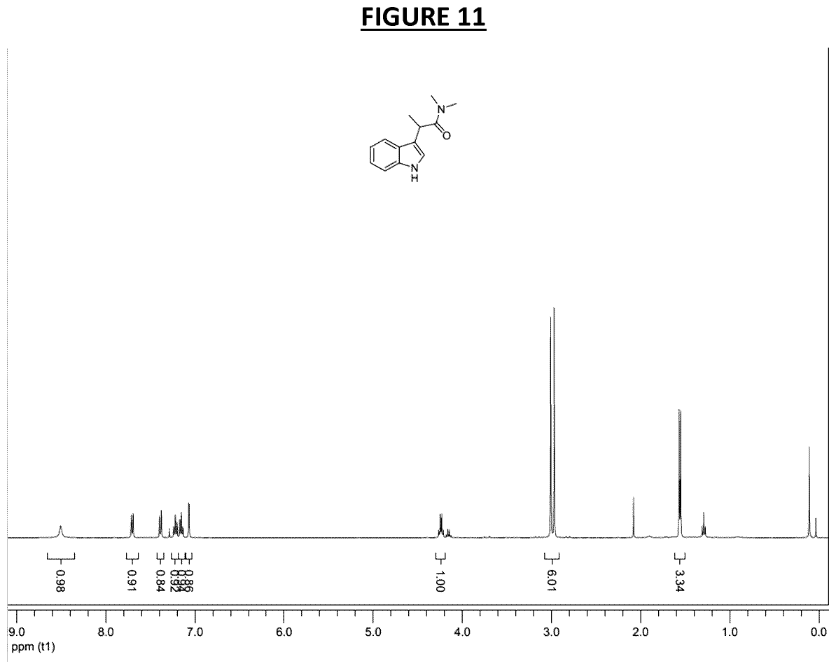

FIG. 11 shows the ¹H NMR spectrum of 2-(1H-indol-3-yl)-N,N-dimethylpropanamide.

### Example 31(xv). 2-(1H-indol-3-yl)-N,N-dimethylbutanamide

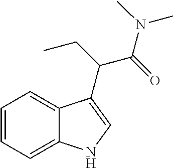 [SMILES: CCC(C(=O)N(C)C)c1c[nH]c2ccccc12]

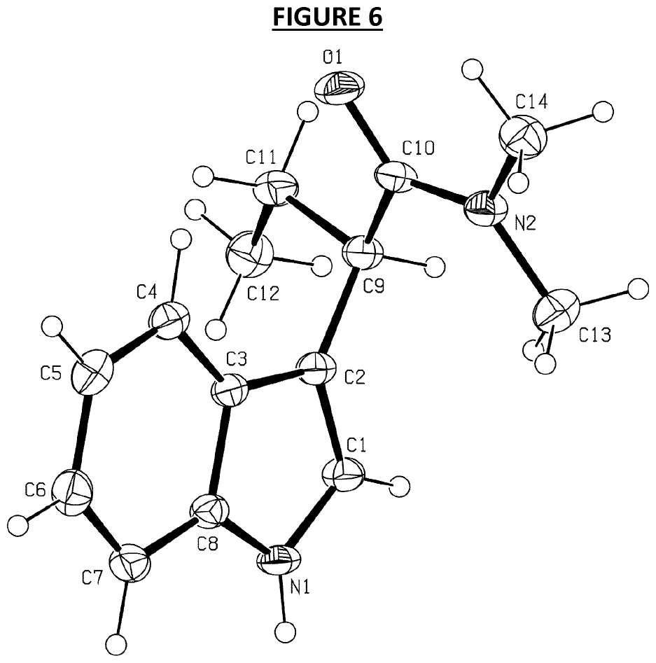

FIG. 6 shows the X-ray crystal structure of 2-(1H-indol-3-yl)-N,N-dimethylbutanamide.

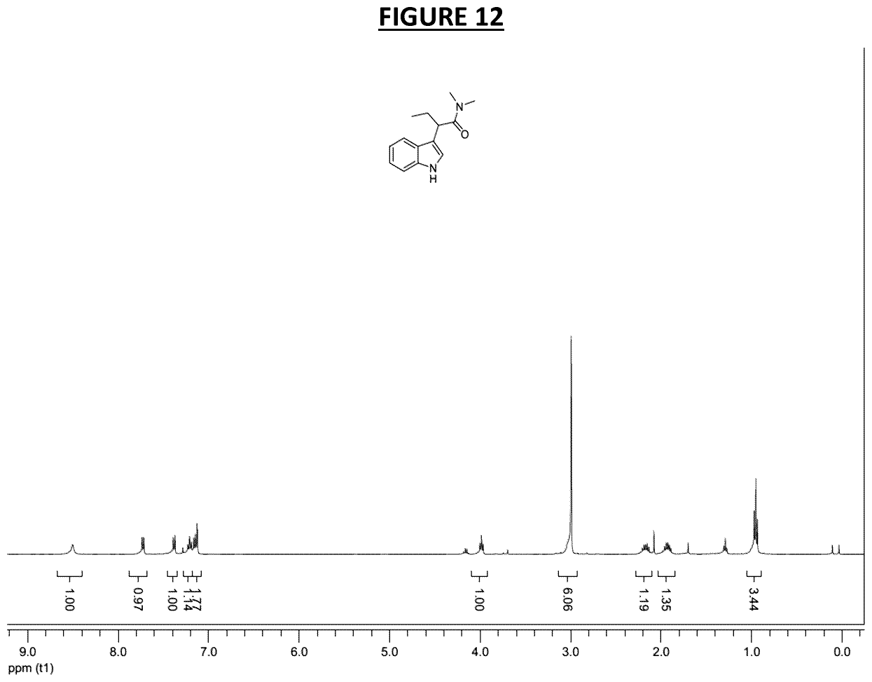

FIG. 12 shows the ¹H NMR spectrum of 2-(1H-indol-3-yl)-N,N-dimethylbutanamide.

### Example 31(xvi). 2-(1H-indol-3-yl)-1-(pyrrolidin-1-yl)ethanone

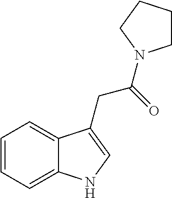 [SMILES: O=C(Cc1c[nH]c2ccccc12)N1CCCC1]

### Example 31(xvii). 2-(1H-indol-3-yl)-1-morpholinoethanone

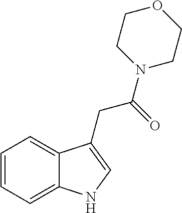 [SMILES: O=C(Cc1c[nH]c2ccccc12)N1CCOCC1]

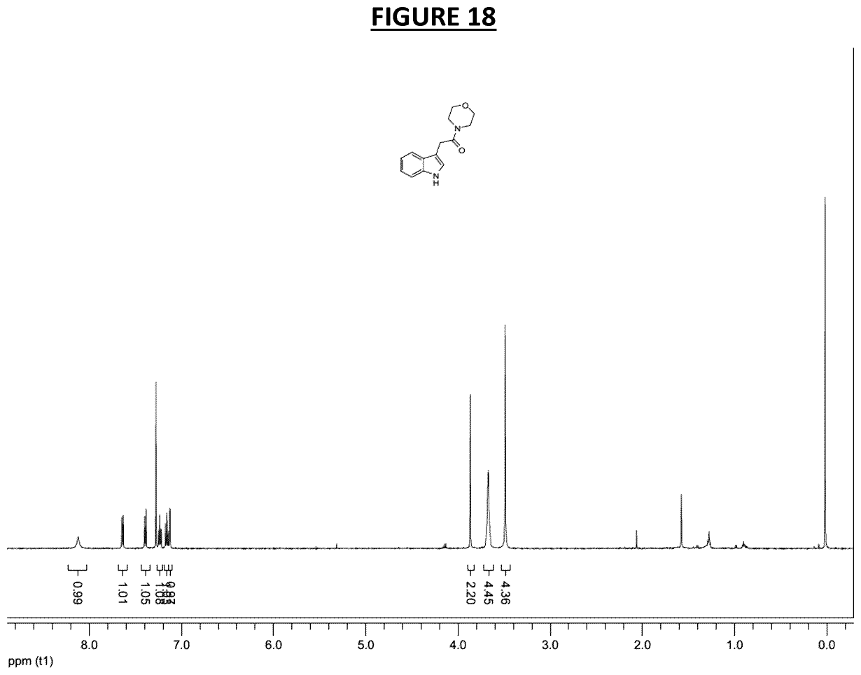

FIG. 18 shows the ¹H NMR spectrum of 2-(1H-indol-3-yl)-1-morpholinoethanone.

### Example 31(xviii). N,N-diethyl-2-(1H-indol-3-yl)acetamide

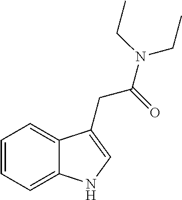 [SMILES: CCN(CC)C(=O)Cc1c[nH]c2ccccc12]

### Example 31(xix). 2-(1H-indol-3-yl)-N,N-diisopropylacetamide

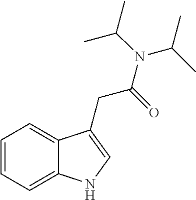 [SMILES: CC(C)N(C(=O)Cc1c[nH]c2ccccc12)C(C)C]

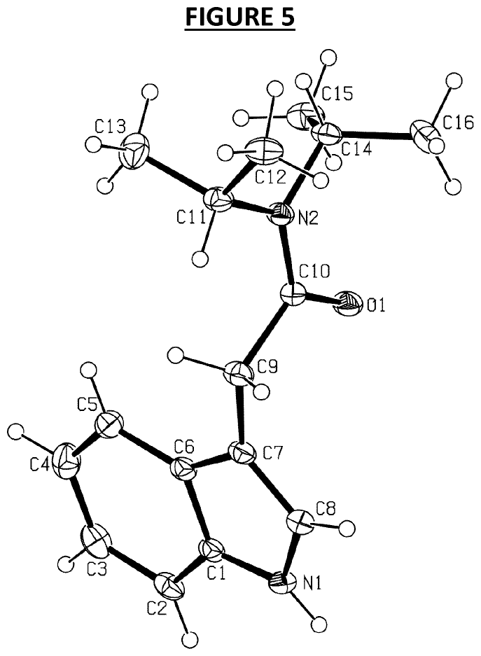

FIG. 5 shows the X-ray crystal structure of 2-(1H-indol-3-yl)-N,N-diisopropylacetamide.

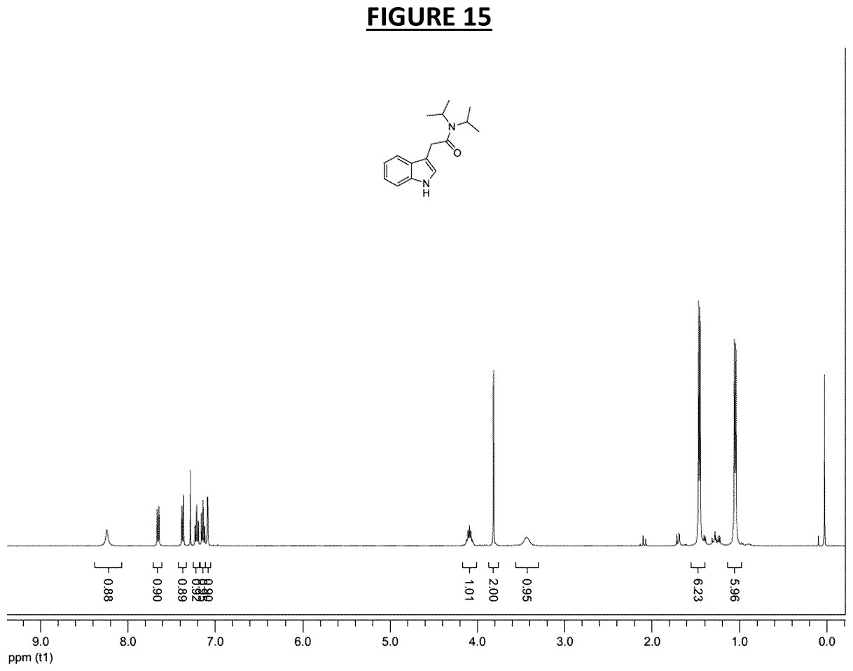

FIG. 15 shows the ¹H NMR spectrum of 2-(1H-indol-3-yl)-N,N-diisopropylacetamide.

### Example 31(xx). N-benzyl-2-(1H-indol-3-yl)-N-methylacetamide

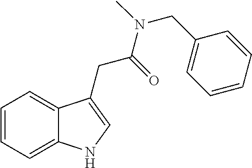 [SMILES: CN(Cc1ccccc1)C(=O)Cc1c[nH]c2ccccc12]

### Example 31(xxi). N,N-dibenzyl-2-(1H-indol-3-yl)acetamide

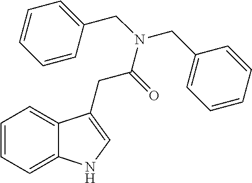 [SMILES: O=C(Cc1c[nH]c2ccccc12)N(Cc1ccccc1)Cc1ccccc1]

### Example 31(xxii). 2-(1H-indol-3-yl)-N,N-bis(methyl-d₃)acetamide

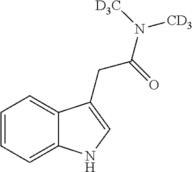 [SMILES: [2H]C([2H])([2H])N(C(=O)Cc1c[nH]c2ccccc12)C([2H])([2H])[2H]]

### Example 31(xxiii). 2-(1H-indol-3-yl)-N,N-di(methyl-¹³C)acetamide

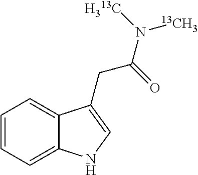

### Example 31(xxiv). 2-(1H-indol-3-yl)-1-(morpholino-d₈)ethanone

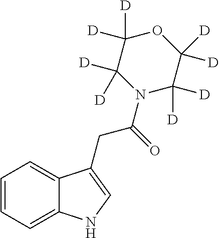 [SMILES: [2H]C1([2H])OC([2H])([2H])C([2H])([2H])N(C(=O)Cc2c[nH]c3ccccc23)C1([2H])[2H]]

### Example 31(xxv). 2-(4-methoxy-1H-indol-3-yl)-N,N-dimethylacetamide

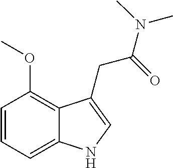 [SMILES: COc1cccc2[nH]cc(CC(=O)N(C)C)c12]

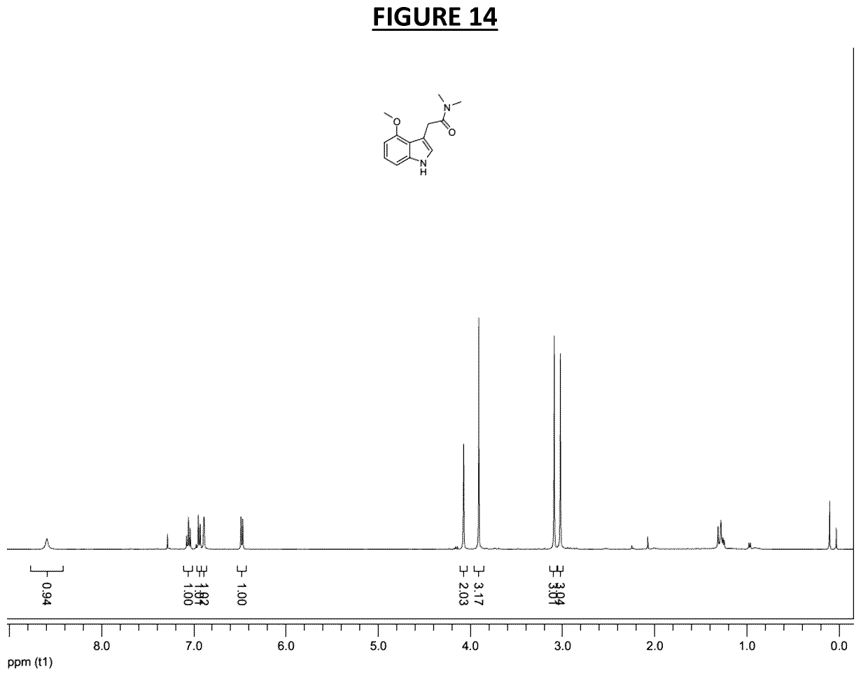

FIG. 14 shows the ¹H NMR spectrum of 2-(4-methoxy-1H-indol-3-yl)-N,N-dimethylacetamide.

### Example 31(xxvi). 2-(4-methoxy-1H-indol-3-yl)-N,N-dimethylpropanamide

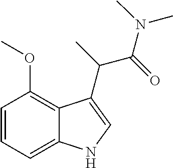 [SMILES: COc1cccc2[nH]cc(C(C)C(=O)N(C)C)c12]

### Example 31(xxvii). 2-(4-methoxy-1H-indol-3-yl)-N,N-dimethylbutanamide

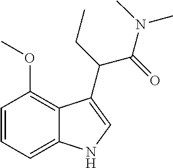 [SMILES: CCC(C(=O)N(C)C)c1c[nH]c2cccc(OC)c12]

### Example 31(xxviii). 2-(4-methoxy-1H-indol-3-yl)-1-(pyrrolidin-1-yl)ethanone

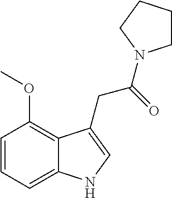 [SMILES: COc1cccc2[nH]cc(CC(=O)N3CCCC3)c12]

### Example 31(xxix). 2-(4-methoxy-1H-indol-3-yl)-1-morpholinoethanone

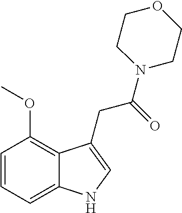 [SMILES: COc1cccc2[nH]cc(CC(=O)N3CCOCC3)c12]

### Example 31(xxx). N,N-diethyl-2-(4-methoxy-1H-indol-3-yl)acetamide

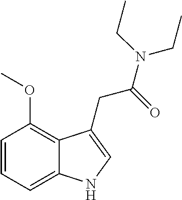 [SMILES: CCN(CC)C(=O)Cc1c[nH]c2cccc(OC)c12]

### Example 31(xxxi). N,N-diisopropyl-2-(4-methoxy-1H-indol-3-yl)acetamide

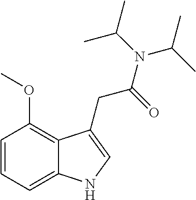 [SMILES: COc1cccc2[nH]cc(CC(=O)N(C(C)C)C(C)C)c12]

### Example 31(xxxii). N-benzyl-2-(4-methoxy-1H-indol-3-yl)-N-methylacetamide

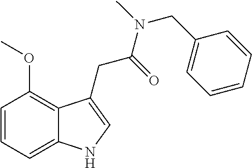 [SMILES: COc1cccc2[nH]cc(CC(=O)N(C)Cc3ccccc3)c12]

### Example 31(xxxiii). N,N-dibenzyl-2-(4-methoxy-1H-indol-3-yl)acetamide

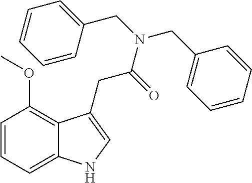 [SMILES: COc1cccc2[nH]cc(CC(=O)N(Cc3ccccc3)Cc3ccccc3)c12]

### Example 31(xxxiv). 2-(4-methoxy-1H-indol-3-yl)-N,N-bis(methyl-d₃)acetamide

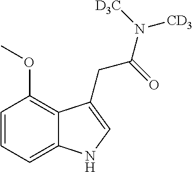 [SMILES: [2H]C([2H])([2H])N(C(=O)Cc1c[nH]c2cccc(OC)c12)C([2H])([2H])[2H]]

### Example 31(xxxv). 2-(4-methoxy-1H-indol-3-yl)-N,N-di(methyl-¹³C)acetamide

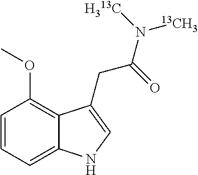 [SMILES: COc1cccc2[nH]cc(CC(=O)N([13CH3])[13CH3])c12]

### Example 31(xxxvi). 2-(4-methoxy-1H-indol-3-yl)-1-(morpholino-d₈)ethanone

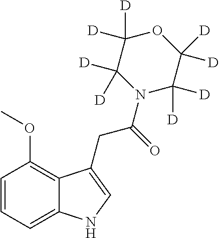 [SMILES: [2H]C1([2H])OC([2H])([2H])C([2H])([2H])N(C(=O)Cc2c[nH]c3cccc(OC)c23)C1([2H])[2H]]

### Example 31(xxxvii). 2-(5-(Methoxy-d₃)-1H-indol-3-yl)-N,N-dimethylacetamide

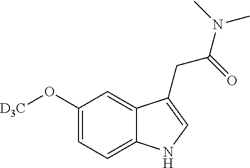 [SMILES: [2H]C([2H])([2H])Oc1ccc2[nH]cc(CC(=O)N(C)C)c2c1]

### Example 31(xxxviii). 2-(4-(Methoxy-d₃)-1H-indol-3-yl)-N,N-dimethylacetamide

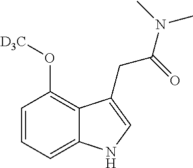 [SMILES: [2H]C([2H])([2H])Oc1cccc2[nH]cc(CC(=O)N(C)C)c12]

### Example 31(xxxix). 2-(5-hydroxy-1H-indol-3-yl)-N,N-dimethylacetamide

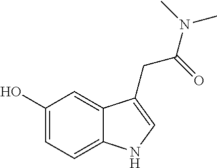 [SMILES: CN(C)C(=O)Cc1c[nH]c2ccc(O)cc12]

### Example 31(xxxx). 2-(4-hydroxy-1H-indol-3-yl)-N,N-dimethylacetamide

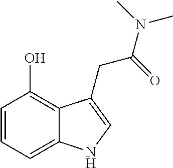 [SMILES: CN(C)C(=O)Cc1c[nH]c2cccc(O)c12]

### Example 31(xxxxi). 3-(5-methoxy-1H-indol-3-yl)-1-methylpyrrolidin-2-one

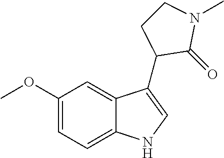

### Example 31(xxxxii). 3-(4-methoxy-1H-indol-3-yl)-1-methylpyrrolidin-2-one

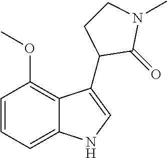 [SMILES: COc1cccc2[nH]cc(C3CCN(C)C3=O)c12]

### Example 31(xxxxiii). 3-(1H-indol-3-yl)-1-methylpyrrolidin-2-one

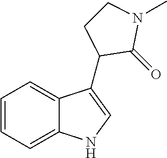 [SMILES: CN1CCC(c2c[nH]c3ccccc23)C1=O]

### Example 32. General Procedure for the Preparation of 2-(1H-indol-3-yl ethanamines

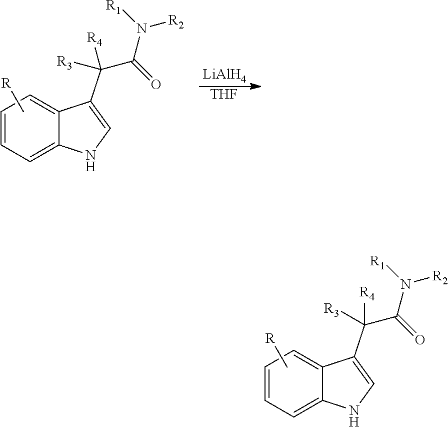

Lithium aluminium hydride solution (1.0 ml of a 1.0 M solution in THF) was added to the 2-(1H-indol-3-yl) acetamide (50 mg) in a Schlenk flask under argon and the mixture stirred for one hour. The solvent was removed, and ether (2 ml) added. Water (2 ml) was added dropwise at 0° C. and the resulting suspension stirred for 30 minutes. The phases were separated, and the ether layer was dried with MgSO₄, filtered and the solvent removed under reduced pressure to give the product.

This procedure was used for the preparation of the products below.

### Example 32(i). 2-(5-methoxy-1H-indol-3-yl)-N,N-dimethylethanamine

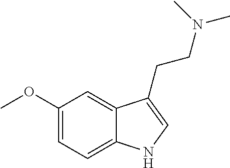

### Example 32(ii). 2-(5-methoxy-1H-indol-3-yl)-N,N-dimethylpropan-1-amine

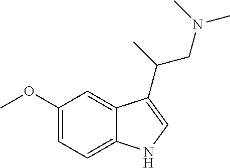

### Example 32(iii). 2-(5-methoxy-1H-indol-3-yl)-N,N-dimethylbutan-1-amine

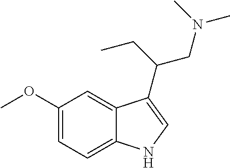

### Example 32(iv). 5-methoxy-3-(2-(pyrrolidin-1-yl)ethyl)-1H-indole

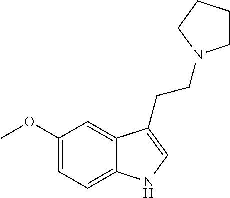 [SMILES: COc1ccc2[nH]cc(CCN3CCCC3)c2c1]

### Example 32(v). 4-(2-(5-methoxy-1H-indol-3-yl)ethyl)morpholine

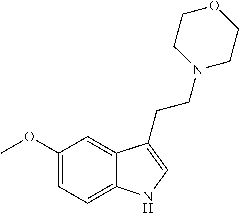 [SMILES: COc1ccc2[nH]cc(CCN3CCOCC3)c2c1]

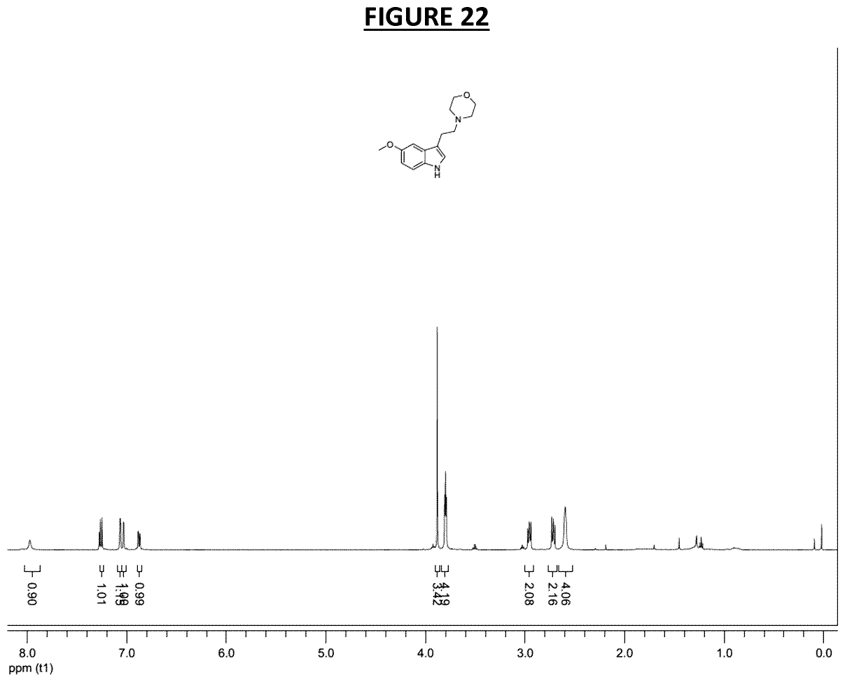

FIG. 22 shows the ¹H NMR spectrum of 4-(2-(5-methoxy-1H-indol-3-yl)ethyl)morpholine.

### Example 32(vi). N-benzyl-2-(5-methoxy-1H-indol-3-yl)-N-methylethanamine

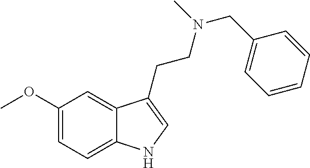 [SMILES: COc1ccc2[nH]cc(CCN(C)Cc3ccccc3)c2c1]

### Example 32(vii). N,N-dibenzyl-2-(5-methoxy-1H-indol-3-yl)ethanamine

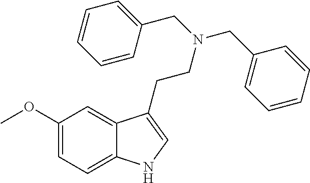 [SMILES: COc1ccc2[nH]cc(CCN(Cc3ccccc3)Cc3ccccc3)c2c1]

### Example 32(viii). 2-(5-methoxy-1H-indol-3-yl)-N,N-bis(methyl-d₃)ethanamine

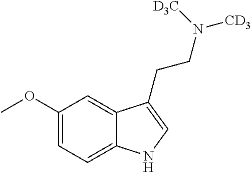 [SMILES: [2H]C([2H])([2H])N(CCc1c[nH]c2ccc(OC)cc12)C([2H])([2H])[2H]]

### Example 32(ix). 2-(5-methoxy-1H-indol-3-yl)-N,N-di(methyl-¹³C)ethanamine

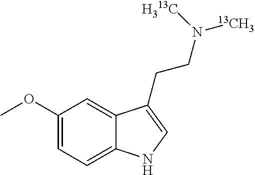

### Example 32(x). 4-(2-(5-methoxy-1H-indol-3-yl)ethyl)morpholine-d₈

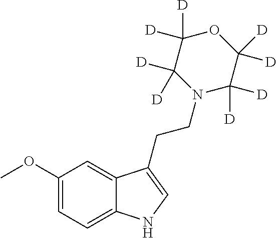 [SMILES: [2H]C1([2H])OC([2H])([2H])C([2H])([2H])N(CCc2c[nH]c3ccc(OC)cc23)C1([2H])[2H]]

### Example 32(xi). 3-(2-(pyrrolidin-1-yl)ethyl)-1H-indole

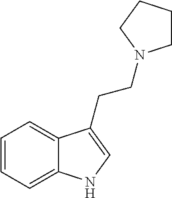 [SMILES: c1ccc2c(CCN3CCCC3)c[nH]c2c1]

### Example 32(xii). 4-(2-(1H-indol-3-yl)ethyl)morpholine

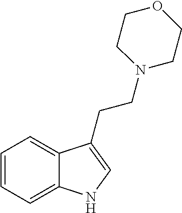 [SMILES: c1ccc2c(CCN3CCOCC3)c[nH]c2c1]

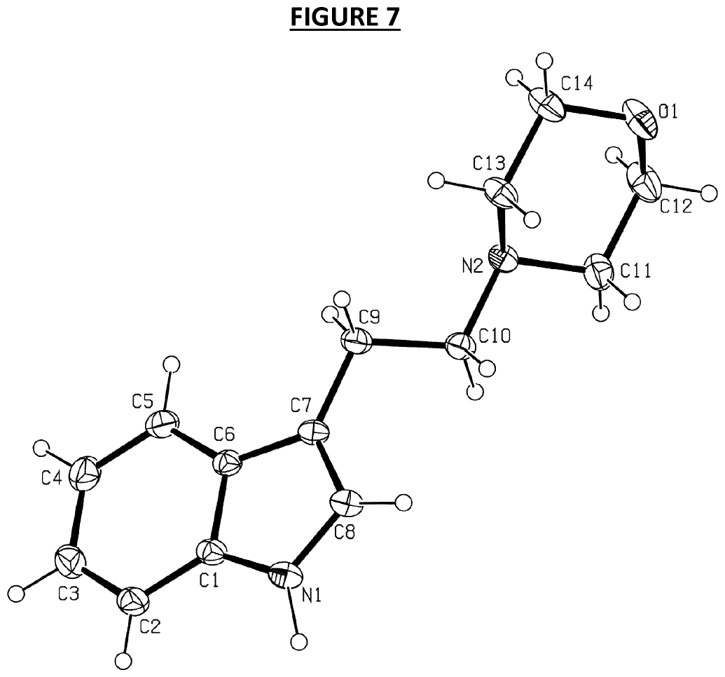

FIG. 7 shows the X-ray crystal structure of 4-(2-(1H-indol-3-yl)ethyl)morpholine.

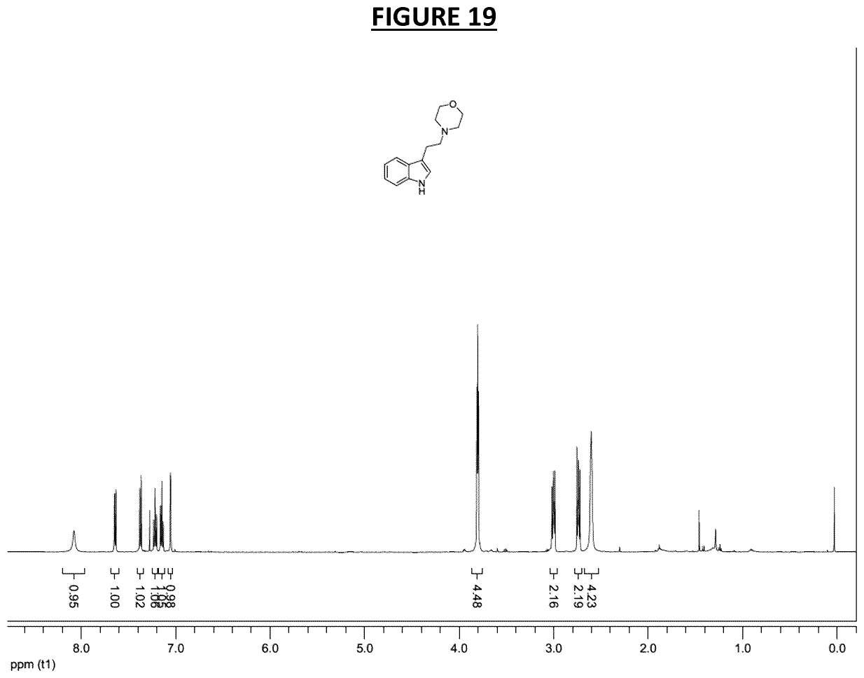

FIG. 19 shows the 1H NMR spectrum of 4-(2-(1H-indol-3-yl)ethyl)morpholine.

### Example 32(xiii). N,N-dibenzyl-2-(1H-indol-3-yl)ethanamine

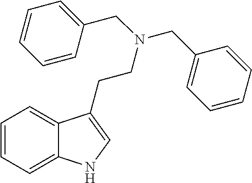 [SMILES: c1ccc(CN(CCc2c[nH]c3ccccc23)Cc2ccccc2)cc1]

### Example 32(xiv). 4-(2-(1H-indol-3-yl)ethyl)morpholine-d₈

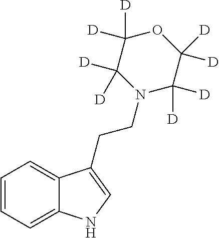 [SMILES: [2H]C1([2H])OC([2H])([2H])C([2H])([2H])N(CCc2c[nH]c3ccccc23)C1([2H])[2H]]

### Example 32(xv). 2-(4-methoxy-1H-indol-3-yl)-N,N-dimethylethanamine

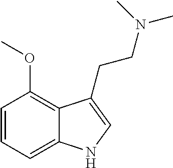 [SMILES: COc1cccc2[nH]cc(CCN(C)C)c12]

### Example 32(xvi). 2-(4-methoxy-1H-indol-3-yl)-N,N-dimethylpropan-1-amine

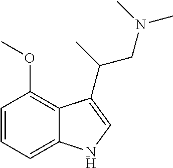 [SMILES: COc1cccc2[nH]cc(C(C)CN(C)C)c12]

### Example 32(xvii). 2-(4-methoxy-1H-indol-3-yl)-N,N-dimethylbutan-1-amine

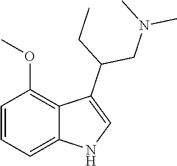 [SMILES: CCC(CN(C)C)c1c[nH]c2cccc(OC)c12]

### Example 32(xviii). 4-methoxy-3-(2-(pyrrolidin-1-yl)ethyl)-1H-indole

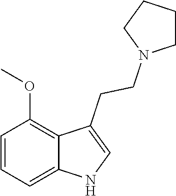 [SMILES: COc1cccc2[nH]cc(CCN3CCCC3)c12]

### Example 32(xix). 4-(2-(4-methoxy-1H-indol-3-yl)ethyl)morpholine

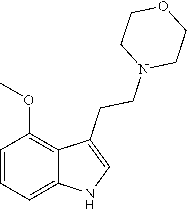 [SMILES: COc1cccc2[nH]cc(CCN3CCOCC3)c12]

### Example 32(xx). N-benzyl-2-(4-methoxy-1H-indol-3-yl)-N-methylethanamine

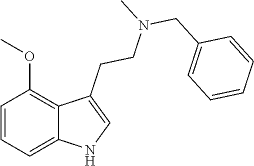 [SMILES: COc1cccc2[nH]cc(CCN(C)Cc3ccccc3)c12]

### Example 32(xxi). N,N-dibenzyl-2-(4-methoxy-1H-indol-3-yl)ethanamine

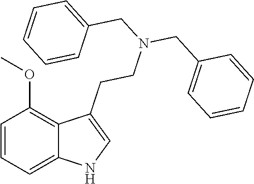 [SMILES: COc1cccc2[nH]cc(CCN(Cc3ccccc3)Cc3ccccc3)c12]

### Example 32(xxii). 2-(4-methoxy-1H-indol-3-yl)-N,N-bis(methyl-d₃)ethanamine

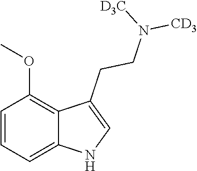 [SMILES: [2H]C([2H])([2H])N(CCc1c[nH]c2cccc(OC)c12)C([2H])([2H])[2H]]

### Example 32(xxiii). 2-(4-methoxy-1H-indol-3-yl)-N,N-di(methyl-¹³C)ethan-1-amine

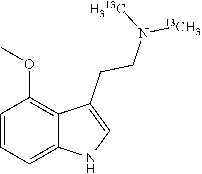 [SMILES: COc1cccc2[nH]cc(CCN([13CH3])[13CH3])c12]

### Example 32(xxiv). 4-(2-(4-methoxy-1H-indol-3-yl)ethyl)morpholine-d₈

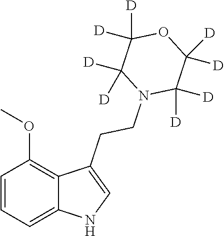 [SMILES: [2H]C1([2H])OC([2H])([2H])C([2H])([2H])N(CCc2c[nH]c3cccc(OC)c23)C1([2H])[2H]]

### Example 32(xxv). 2-(5-(methoxy-d₃)-1H-indol-3-yl)-N,N-dimethylethan-1-amine

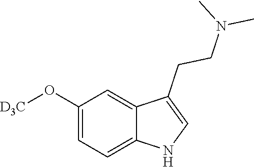 [SMILES: [2H]C([2H])([2H])Oc1ccc2[nH]cc(CCN(C)C)c2c1]

### Example 32(xxvi). 2-(4-(methoxy-d₃)-1H-indol-3-yl)-N,N-dimethylethan-1-amine

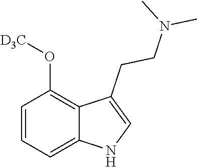 [SMILES: [2H]C([2H])([2H])Oc1cccc2[nH]cc(CCN(C)C)c12]

### Example 32(xxvii). 3-(2-(dimethylamino)ethyl)-1H-indol-5-ol

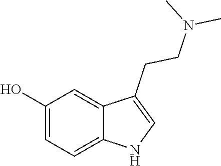

### Example 33. General Procedure for the LiAlD₄ reduction of 2-(1H-indol-3-yl) acetamides

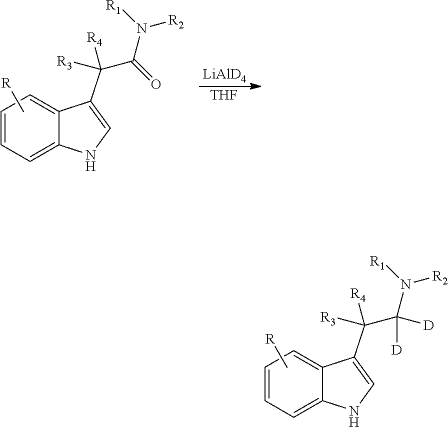

Lithium aluminium deuteride solution (1.0 ml of a 1.0 M solution in THF) was added to the 2-(1H-indol-3-yl) acetamide (50 mg) in a Schlenk flask under argon and the mixture stirred for one hour. The solvent was removed, and ether (2 ml) added. Water (2 ml) was added dropwise at 0° C. and the resulting suspension stirred for 30 minutes. The phases were separated, and the ether layer was dried with MgSO₄, filtered and the solvent removed under reduced pressure to give the product.

This procedure was used for the preparation of the products below.

### Example 33(i). 2-(5-methoxy-1H-indol-3-yl)-N,N-dimethylethan-1-amine-1,1-d₂

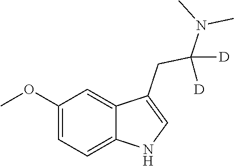

### Example 33(ii). 2-(5-methoxy-1H-indol-3-yl)-N,N-dimethylpropan-1-amine-1,1-d₂

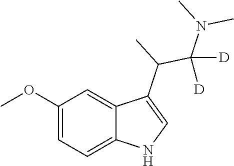

### Example 33(iii). 2-(5-methoxy-1H-indol-3-yl)-N,N-dimethylbutan-1-amine-1,1-d₂

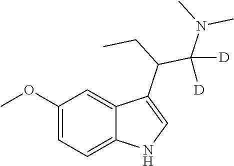

### Example 33(iv). 5-methoxy-3-(2-(pyrrolidin-1-yl)ethyl-2,2-d₂)-1H-indole

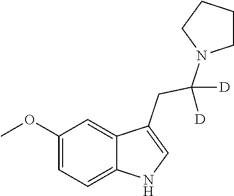 [SMILES: [2H]C([2H])(Cc1c[nH]c2ccc(OC)cc12)N1CCCC1]

### Example 33(v). 4-(2-(5-methoxy-1H-indol-3-yl)ethyl-1,1-d₂)morpholine

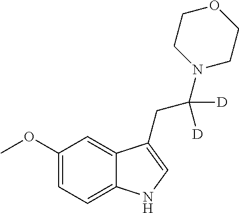 [SMILES: [2H]C([2H])(Cc1c[nH]c2ccc(OC)cc12)N1CCOCC1]

### Example 33(vi). 3-(2-(pyrrolidin-1-yl)ethyl-2,2-d₂)-1H-indole

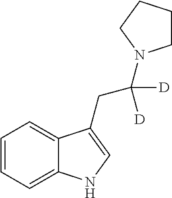 [SMILES: [2H]C([2H])(Cc1c[nH]c2ccccc12)N1CCCC1]

### Example 33(vii). 4-(2-(1H-indol-3-yl)ethyl-1,1-d₂)morpholine

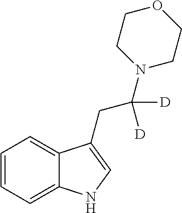 [SMILES: [2H]C([2H])(Cc1c[nH]c2ccccc12)N1CCOCC1]

### Example 33(viii). 2-(4-methoxy-1H-indol-3-yl)-N,N-dimethylethan-1-amine-1,1-d₂

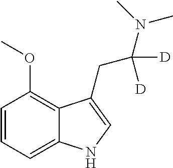 [SMILES: [2H]C([2H])(Cc1c[nH]c2cccc(OC)c12)N(C)C]

### Example 33(ix). 2-(4-methoxy-1H-indol-3-yl)-N,N-dimethylpropan-amine-1,1-d₂

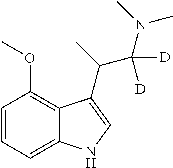 [SMILES: [2H]C([2H])(C(C)c1c[nH]c2cccc(OC)c12)N(C)C]

### Example 33(x). 2-(4-methoxy-1H-indol-3-yl)-N,N-dimethylbutan-1-amine-1,1-d₂

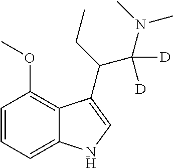 [SMILES: [2H]C([2H])(C(CC)c1c[nH]c2cccc(OC)c12)N(C)C]

### Example 33(xi). 4-methoxy-3-(2-(pyrrolidin-1-yl)ethyl-2,2-d₂)-1H-indole

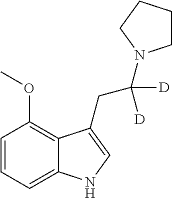 [SMILES: [2H]C([2H])(Cc1c[nH]c2cccc(OC)c12)N1CCCC1]

### Example 33(xii). 4-(2-(4-methoxy-1H-indol-3-yl)ethyl-1,1-d₂)morpholine

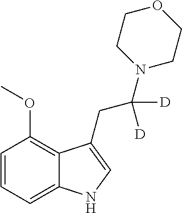 [SMILES: [2H]C([2H])(Cc1c[nH]c2cccc(OC)c12)N1CCOCC1]

### Example 33(xiii). 3-(2-(dimethylamino)ethyl-2,2-d₂)-1H-indol-5-ol

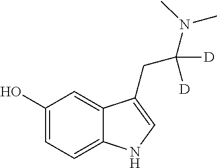 [SMILES: [2H]C([2H])(Cc1c[nH]c2ccc(O)cc12)N(C)C]

While the foregoing disclosure has been described in some detail for purposes of clarity and understanding, it will be appreciated by one skilled in the art, from a reading of the disclosure that various changes in form and detail can be made without departing from the true scope of the disclosure in the appended claims.

All publications, patents, and patent applications are herein incorporated by reference in their entirety to the same extent as if each individual publication, patent or patent application was specifically and individually indicated to be incorporated by reference in its entirety.

1. A process for the preparation of compounds of Formula (III): 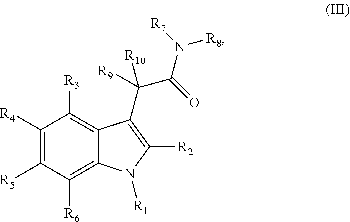
comprising contacting a compound of Formula (I): 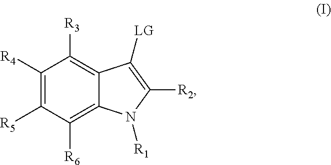
with a compound of Formula (II): 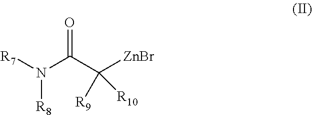
in the presence of a catalyst,
wherein,
R₁ is hydrogen, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, —(C═O)—O—(C₁-C₂₀)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₂₀)-alkyl, (C₂-C₂₀)-alkenyl, (C₂-C₂₀)-alkynyl, (C₃-C₂₀)-cycloalkyl, or (C₆-C₂₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl;
R₂ to R₁₀ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl;
LG represents any suitable leaving group.

2. The process according to claim 1, wherein one or more of the carbon-12 atoms in a compound of Formula (III) are replaced with carbon-13.

3. The process according to claim 1, wherein the compound of Formula (III) is achiral.

4. The process according to claim 1, wherein the compound of Formula (III) is chiral.

5. The process according to claim 1, wherein the catalyst comprises transition metal salts and complexes.

6. The process according to claim 5, wherein the transition metal salts and complexes comprises palladium, nickel, iron, ruthenium, cobalt, rhodium, iridium or copper.

7. The process according to claim 1, wherein R₁ represents hydrogen, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, —(C═O)—O—(C₁-C₁₀)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₁₀)-alkyl, (C₂-C₁₀)-alkenyl, (C₂-C₁₀)-alkynyl, (C₃-C₁₀)-cycloalkyl, or (C₆-C₁₀)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

8. The process according to claim 7, wherein R₁ represents hydrogen, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, —(C═O)—O—(C₁-C₆)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₆)-alkyl, (C₂-C₆)-alkenyl, (C₂-C₆)-alkynyl, (C₃-C₇)-cycloalkyl, or (C₆)-aryl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

9. The process according to claim 8, wherein R₁ represents hydrogen, (C₁-C₆)-alkyl, (C₂-C₆)-alkenyl, (C₂-C₆)-alkynyl, (C₃-C₇)-cycloalkyl, phenyl, or —C(═O)—(C₁-C₆)-alkyl, each of which are optionally substituted with halogen, OH, optionally substituted phenyl or (C₁-C₆)-alkyl, and one or more of the carbon atoms in the alkyl, alkenyl, alkynyl, cycloalkyl, aryl, heteroaryl or acyl groups of R₁ is optionally replaced with a heteroatom selected from the group consisting of O, S, N, P and Si, which, where possible, is optionally substituted with one or more groups selected from halo, OH, optionally substituted phenyl or (C₁-C₆)-alkyl.

10. The process according to claim 1, wherein R₂ to R₁₀ represent hydrogen, deuterium, (C₁-C₁₀)-alkyl, (C₂-C₁₀)-alkenyl, (C₂-C₁₀)-alkynyl, (C₃-C₁₀)-cycloalkyl, (C₆-C₁₀)-aryl, (C₅-C₁₀)-heteroaryl, —C(═O)—(C₁-C₁₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

11. The process according to claim 10, wherein R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, (C₅-C₆)-heteroaryl, —C(═O)—(C₁-C₆)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

12. The process according to claim 11, wherein R₂ to R₁₀ represent hydrogen, deuterium, (C₁-C₆)-alkyl, (C₂-C₆)-alkenyl, (C₂-C₆)-alkynyl, (C₃-C₇)-cycloalkyl, (C₆)-aryl, or (C₅-C₆)-heteroaryl.

13. The process according to claim 1, wherein LG is an anionic leaving group.

14. The process according to claim 13, wherein LG is a halide group, or a sulphonate.

15. A process for the preparation of compounds of Formula (IV): 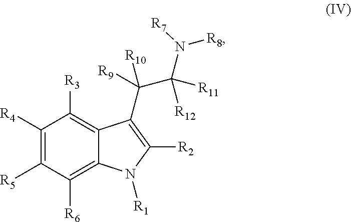
by contacting a compound of Formula (III), 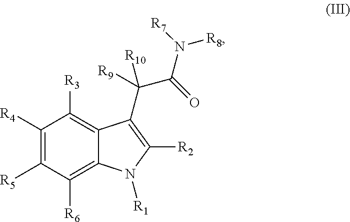
with a hydrogen or deuterium source optionally in the presence of a catalyst wherein,
R₁ to R₁₀ are as defined in claim 1; and
R₁₁ to R₂ represent hydrogen or deuterium.

16. (canceled)

17. A process according to claim 15, wherein the compound of Formula (IV) is chiral.

18. A process according to claim 15, wherein one or more of the carbon-12 atoms in a compound of Formula (IV) are replaced with carbon-13.

19. A process according to claim 15, for the preparation of a compound of Formula (IV) by contacting a compound of Formula (III) with a hydrogen or deuterium source in the absence of a catalyst.

20. A process according to claim 15, wherein the hydrogen or deuterium source is a borohydride, a borodeuteride, an aluminohydride, an aluminodeuteride, a silane, a borane, hydrogen gas or deuterium gas.

21. A Compound of Formula (V), Formula (VI), Formula (VII) or Formula (VIII): 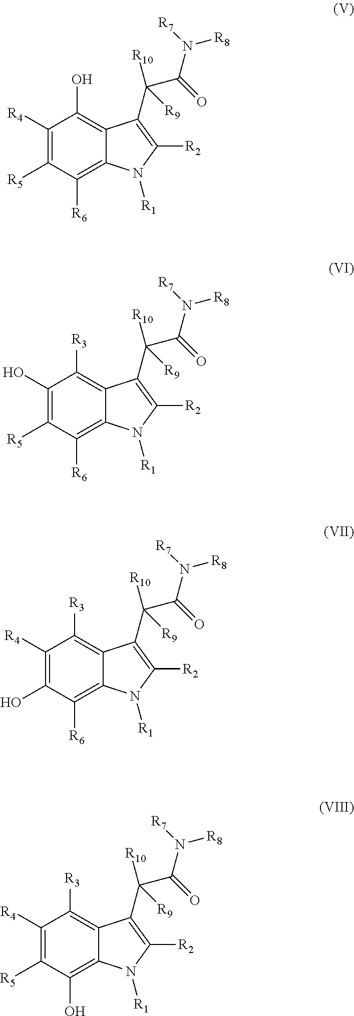
wherein,
R₁ represents hydrogen, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, —(C═O)—O—(C₁-C₂₀)-alkyl, OR^{c}, or NR^{c}₂, each of which are optionally substituted with halogen, OH, optionally substituted phenyl or (C₁-C₆)-alkyl, and wherein R^{c} is hydrogen, (C₁-C₂₀)-alkyl, (C₂-C₂₀)-alkenyl, (C₂-C₂₀)-alkynyl, (C₃-C₂₀)-cycloalkyl, or (C₆-C₂₀)-aryl;
R₂ to R₁₀ represent hydrogen, deuterium, (C₁-C₂₀)-alkyl, (C₂-C₂₀)-alkenyl, (C₂-C₂₀)-alkynyl, (C₃-C₂₀)-cycloalkyl, (C₆-C₂₀)-aryl, (C₅-C₂₀)-heteroaryl, —C(═O)—(C₁-C₂₀)-alkyl, or two adjacent or geminal groups are bonded together to form an optionally substituted ring, each of which is optionally substituted with halogen, OH, or (C₁-C₆)-alkyl, and one or more of the carbon atoms in the alkyl, alkenyl, alkynyl, cycloalkyl, aryl, heteroaryl or acyl groups of R₂ to R₁₀ is optionally replaced with a heteroatom selected from the group consisting of O, S, N, P and Si, which is optionally substituted with one or more groups selected from halogen, OH, and (C₁-C₆)-alkyl.

22.-26. (canceled)
